(19) United States
(12) Patent Application Publication
(10) Pub. No.: US 2022/0372573 A1
(43) Pub. Date: Nov. 24, 2022

(54) METHODS AND SYSTEMS FOR DETECTION OF KIDNEY DISEASE OR DISORDER BY GENE EXPRESSION ANALYSIS

(71) Applicants: Impetus Bioscientific Inc., Millbrae, CA (US); The Regents of the University of California, Oakland, CA (US)

(72) Inventors: Ying Bing Liu, Millbrae, CA (US); Helen Zhao, Brooklyn, NY (US); Weiming Ruan, San Francisco, CA (US); Alan H. Wu, Palo Alto, CA (US); Elizabeth J. Murphy, San Francisco, CA (US)

(21) Appl. No.: 17/324,383

(22) Filed: May 19, 2021

(51) Int. Cl. C12Q 1/6883; G16B 30/00; G16B 20/20

(52) U.S. Cl. CPC C12Q 1/6883; G16B 30/00; G16B 20/20; C12Q 2600/158

(57) ABSTRACT

The present disclosure provides methods and systems directed to detection of kidney disease or disorder. A method for processing or analyzing a bodily sample of a subject may comprise (a) analyzing the bodily sample to yield a data set comprising one or more levels of gene expression products in the bodily sample, which one or more levels of gene expression products correspond to a set of genes associated with a kidney disease or disorder; (b) computer processing the data set to determine a presence or an elevated risk of the kidney disease or disorder in the subject; and (c) electronically outputting a report that identifies the presence or the elevated risk of the kidney disease or disorder in the subject.

### BACKGROUND

The International Society of Nephrology estimates that 850 million people worldwide are affected by kidney disease. Diabetic nephropathy (DN) is a major cause of kidney disease and is the most common cause of end-stage renal disease (ESRD). In addition, DN is linked to higher cardiovascular and all-cause morbidity and mortality, so timely diagnosis and treatment are critical. Diabetic nephropathy is a diabetic kidney disease that generally refers to kidney damage that results from having high blood glucose levels due to diabetes. Diabetic nephropathy progresses slowly. With efficient early treatment, one can slow or even stop the progression of the disease. DN may be associated with measurable biomarkers, such as albuminuria and/or low eGFR, in bodily samples of subjects; however, these biomarkers may be non-specific to DN, and may be attributable to other disease or disorders, such as diabetes, hypertension, IgA nephropathy, membranous nephropathy, lupus nephritis, minimal change disease, rheumatoid arthritis, medicine such as NSAIDs use, smoking, excessive alcohol use, drugs of abuse, obesity, urinary tract infection, kidney stone, benign prostate hyperplasia (BPH), etc. Further, patients with controlled diabetes may also have diabetic nephropathy, whereas poorly controlled diabetic patients may have little kidney damage. Moreover, patients with diabetic nephropathy may also have other type(s) of kidney disease or disorder.

### SUMMARY

DN is an under-diagnosed and mis-diagnosed disease among patients. For example, DN may be under-diagnosed because diagnostics methods such as renal biopsy can be risky (e.g., with a 1.8% mortality rate), expensive, and time-consuming; therefore, many patients opt out of such diagnostic testing. As another example, DN may be mis-diagnosed because diagnostics methods may lack adequate sensitivity and specificity. For example, a urinary albumin assay may be insensitive and non-specific. As another example, imaging tests such as X-rays and ultrasound diagnostics to check the structure and size of a subject's kidney may be time-consuming and indirect, and other panel tests such as urinary sediment test, urine protein electrophoresis (UPEP), serum protein electrophoresis (SPEP), urine blood test, antinuclear antibody (ANA) test, HBV test, HCV test, HIV test may provide limited information, and therefore are indirect and inefficient. In vitro diagnostic techniques, such as those based on proteomics, genomics, and protein biomarkers, may face challenges in accurately detecting, assessing, and monitoring kidney disease or disorder at high sensitivity, specificity, and accuracy. It may also be difficult to identify early diabetic changes, since the only biomarker typically used is albuminuria; however, albuminuria, especially at low levels, may be confounded with many other factors such as hypertension, smoking, alcohol use, drug abuse, obesity, infection, obstruction, etc. Therefore, many patients may miss the best opportunity for early medical intervention or may receive treatment that is unrelated to the cause of the disease. Receiving such unnecessary and/or ineffective treatment may be expensive, time consuming, and cause delays in providing other effective treatments to patients.

Recognizing the need for improved methods for detecting, assessing, and monitoring kidney disease or disorder (e.g., DN) that are fast, inexpensive, non-invasive, and highly sensitive, specific, and accurate, the present disclosure provides methods, systems, and kits for detecting kidney disease or disorders by processing biological samples (e.g., tissue samples, cell samples, and/or bodily fluid samples) obtained from or derived from a subject. For example, nucleic acids, proteins, or cells of biological samples may be analyzed. Biological samples obtained from subjects may be analyzed to measure a presence, absence, or relative assessment of the kidney disease or disorder. The analysis may be performed at a set of genomic regions, such as kidney disease-associated genes or genomic loci. The subjects may include subjects with kidney disease or disorder (e.g., kidney disease or disorder patients) and subjects without kidney disease or disorder (e.g., normal or healthy controls).

Methods of the present disclosure may present numerous advantages over current methods, including: ease, safety, and non-invasiveness of sample collection from subjects, possibility of repeated assays, the use of urine samples which have kidney cells suitable for analysis, a direct method of analysis of kidney injury, suitability for monitoring disease progression and treatment efficacy, suitability for sample collection in a home setting, the ability to perform testing without detailed medical history of a subject, and the ability to detect early diabetic changes (e.g., for asymptomatic subjects).

Using methods and systems of the present disclosure, kidney disease or disorder can be accurately detected using an assay with high sensitivity and specificity in biological samples (e.g., urine samples). The urine-based assay can apply machine learning algorithm to analyze a set of biomarkers to accurately distinguish kidney disease or disorder samples from control samples across various stages (e.g., early-stage, mid-stage, or late-stage) of kidney disease or disorder. Further, the urine-based assay may offer high specificity, thereby facilitating the non-invasive application of kidney disease or disorder associated biomarkers for treatment monitoring of kidney disease or disorder patients. In addition, the urine-based assay may offer higher sensitivity and specificity than those of renal biopsy, currently considered as the gold standard for definitive diagnosis of kidney diseases.

In an aspect, the present disclosure provides a method for processing or analyzing a bodily sample of a subject, comprising: (a) analyzing the bodily sample to yield a data set comprising one or more levels of gene expression products in the bodily sample, which one or more levels of gene expression products correspond to a set of genes associated with a kidney disease or disorder; (b) computer processing the data set from (a) to determine a presence or an elevated risk of the kidney disease or disorder in the subject at an accuracy of at least about 80%; and (c) electronically outputting a report that identifies the presence or the elevated risk of the kidney disease or disorder in the subject determined in (b).

In some embodiments, the bodily sample is selected from the group consisting of: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample. In some embodiments, the bodily sample is the urine sample. In some embodiments, the urine sample is a fresh urine sample. In some embodiments, the urine sample is a frozen sample (e.g., freshly frozen sample). In some embodiments, the urine sample is a preserved urine sample (e.g., stored at room temperature in preservatives to prevent degradation).

In some embodiments, (a) comprises reverse transcribing ribonucleic acid (RNA) molecules obtained or derived from the bodily sample to yield complementary deoxyribonucleic acid (cDNA) molecules, and sequencing at least a portion of the cDNA molecules to yield sequencing reads. In some embodiments, sequencing reads are mapped to a reference sequence (e.g., a reference genome such as a human genome) to yield the data set, wherein the data set comprises counts of gene transcripts. In some embodiments, each count of a transcript is indicative of a gene expression event.

In some embodiments, (a) comprises reverse transcribing ribonucleic acid (RNA) molecules obtained or derived from the bodily sample to yield complementary deoxyribonucleic acid (cDNA) molecules, and assaying at least a portion of the cDNA molecules by Real-time polymerase chain reaction (RT-PCR, also called qPCR) to yield the data set. In some embodiments, the data set comprises cycle threshold (Ct) values, which are inversely proportional to gene expression levels.

In some embodiments, (a) comprises reverse transcribing ribonucleic acid (RNA) molecules obtained or derived from the bodily sample to yield complementary deoxyribonucleic acid (cDNA) molecules, and assaying at least a portion of the cDNA molecules by microarray analysis (e.g. Affymetrix Microarray) to yield the data set. In some embodiments, the data set comprises counts of gene transcripts. In some embodiments, each count is indicative of a gene expression event. In some embodiments, (a) comprises hybridizing ribonucleic acid (RNA) molecules obtained or derived from the bodily sample using specific probe sets, and assaying the hybridized RNA molecules using a Luminex platform to yield the data set. In some embodiments, the data set comprises counts of gene transcripts.

In some embodiments, (a) comprises selectively enriching at least a portion of the cDNA molecules or the RNA molecules for a set of genomic loci associated with the kidney disease or disorder. In some embodiments, (a) comprises amplifying at least a portion of the cDNA molecules or the RNA molecules. In some embodiments, (a) comprises aligning at least a portion of the sequencing reads to a reference sequence. In some embodiments, (a) comprises generating counts of gene transcripts. In some embodiments, the reference sequence is at least a portion of a human reference genome. In some embodiments, the counts of gene transcripts are normalized to generate normalized counts of gene transcripts for downstream differential gene expression analysis. In some embodiments, the normalizing comprises determining CPM (counts per million), TPM (transcripts per kilobase million), or RPKM/FPKM (reads/fragments per kilobase of exon per million reads/fragments mapped).

In some embodiments, the kidney disease or disorder is selected from the group consisting of: early-stage kidney disease, mid-stage kidney disease, late-stage kidney disease, end-stage kidney disease, asymptomatic kidney disease, diabetic nephropathy, hypertensive nephropathy, IgA nephropathy, membranous nephropathy, minimal change disease, focal segmental glomerulosclerosis (FSGS), NSAIDs induced nephrotoxicity, thin basement membrane nephropathy, amyloidosis, ANCA vasculitis related to endocarditis and other infections, cardiorenal syndrome, IgG4 nephropathy, interstitial nephritis, lithium nephrotoxicity, lupus nephritis, multiple myeloma, polycystic kidney disease, pyelonephritis (kidney infection), renal artery stenosis, renal cyst, rheumatoid arthritis-associated renal disease, and kidney stone. In some embodiments, the kidney disease or disorder is diabetic nephropathy. In some embodiments, diabetic nephropathy is early-stage diabetic nephropathy. In some embodiments, the subject is asymptomatic for the diabetic nephropathy.

In some embodiments, (b) comprises using a trained algorithm to process the data set. In some embodiments, the trained algorithm comprises a trained machine learning algorithm. In some embodiments, the trained machine learning algorithm is selected from the group consisting of: a support vector machine (SVM), a naive Bayes classification, a linear regression, a quantile regression, a logistic regression, a nonlinear regression, a random forest, a neural network, an ensemble learning method, a boosting algorithm, an AdaBoost algorithm, a recursive feature elimination algorithm (RFE), and any combination thereof. In some embodiments, the trained machine learning algorithm comprises the recursive feature elimination (RFE) algorithm. In some embodiments, the trained machine learning algorithm is trained with a plurality of training samples comprising a first set of bodily samples from subjects having the type of kidney disease or disorder of interest (e.g. diabetic nephropathy) and a second set of bodily samples from subjects having no kidney disease or disorder, wherein the first set of bodily samples and the second set of bodily samples are different from the bodily sample of the subject. In some embodiments, the trained machine learning algorithm is trained with a plurality of training samples comprising a first set of bodily samples from subjects having the type of kidney disease or disorder of interest (e.g. diabetic nephropathy) and a second set of bodily samples from subjects having other types of kidney disease or disorder, wherein the first set of bodily samples and the second set of bodily samples are different from the bodily sample of the subject.

In some embodiments, (a) comprises comparing one or more levels of gene expression products to a reference. In some embodiments, the reference corresponds to a set of gene expression products from a first set of bodily samples from subjects having the type of kidney disease or disorder of interest (e.g. diabetic nephropathy) and/or a second set of bodily samples from subjects having no kidney disease or disorder. In some embodiments, the reference corresponds to a set of gene expression products from a first set of bodily samples from subjects having the type of kidney disease or disorder of interest (e.g. diabetic nephropathy) and/or a second set of bodily samples from subjects having other types of kidney disease or disorder.

In some embodiments, the method further comprises detecting the presence or the elevated risk of the kidney disease or disorder in the subject at a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at an Area Under the Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99.

In some embodiments, the method further comprises identifying a clinical intervention for the subject based at least in part on the presence or the elevated risk of the kidney disease or disorder determined in (b). In some embodiments, the clinical intervention is selected from the group consisting of: a drug treatment, intensive glycemic control, high blood pressure control, lower high cholesterol, foster bone health, diet control, lifestyle changes, weight loss, exercise, tobacco cessation, manage alcohol intake, reduce/quit drugs of abuse, and avoiding NSAIDs. In some embodiments, the drug relies on the blockage of the renin-angiotensin aldosterone system (RAAS, e.g., angiotensin-converting enzyme (ACE) inhibitors and angiotensin II receptor blockers (ARBs)). In some embodiments, the drug is an experimental therapy, such as a drug targeting vasculature/hemodynamic effects, a drug-targeting inflammation, and a drug targeting oxidative stress.

In some embodiments, (b) comprises analyzing a first set of genes that differentially distinguishes between a first kidney disease or disorder and negative (NEG) subjects and a second set of genes that differentially distinguishes between the first kidney disease or disorder and a second kidney disease or disorder. In some embodiments, (b) comprises analyzing a first set of genes that differentially distinguishes between diabetic nephropathy (DN) and negative (NEG) subjects and a second set of genes that differentially distinguishes between DN and other chronic kidney diseases (CKD). In some embodiments, the first set of genes is selected from the group of genes listed in Table 3 and Table 5, and wherein the second set of genes is selected from the group of genes listed in Table 4 and Table 6. In some embodiments, (b) comprises generating a first DN vs. NEG score based on the first set of genes and a second DN vs. CKD score based on the second set of genes. In some embodiments, the first DN vs. NEG score is indicative of glomerular injury when greater than 0.5, or is indicative of tubular injury when less than 0.5. In some embodiments, (b) comprises analyzing different male-specific or female-specific sets of genes based on a gender of the subject.

In some embodiments, the method further comprises removing at least a subset of negative (NEG) subjects who have a pre-determined characteristic (e.g., are obese, are morbidly obese, are nicotine dependent, are alcohol dependent, are drugs-of-abuse dependent, have kidney stone, have severe hypertension, have urinary tract infection, have heart diseases, have hepatitis B, have hepatitis C, have HIV, have psoriasis, have rheumatoid arthritis, use NSAIDs, etc.), and optionally replacing with additional NEG subjects who do not have the pre-determined characteristic, to generate a modified set of NEG-X subjects (e.g., where X is the pre-determined characteristic). In some embodiments, if the DN vs. NEG-X score is much higher than the DN vs. NEG score (e.g., >0.1), that is indicative of the kidney damage being a result of the pre-determined characteristic X.

In some embodiments, the method further comprises removing at least a subset of other chronic kidney disease (CKD) subjects who have a pre-determined characteristic (e.g., are obese, are morbidly obese, are nicotine dependent, are alcohol dependent, are drugs-of-abuse dependent, have kidney stone, have severe hypertension, have urinary tract infection, have heart diseases, have hepatitis B, have hepatitis C, have HIV, have psoriasis, have rheumatoid arthritis, use NSAIDs, have IgA nephropathy, have membranous nephropathy, have minimal change disease, have focal segmental glomerulosclerosis (FSGS), have thin basement membrane nephropathy, have amyloidosis, have ANCA vasculitis related to endocarditis and other infections, have cardiorenal syndrome, have IgG4 nephropathy, have interstitial nephritis, have lithium nephrotoxicity, have lupus nephritis, have multiple myeloma, have polycystic kidney disease, have pyelonephritis (kidney infection), have renal artery stenosis, have renal cyst, have rheumatoid arthritis-associated renal disease, etc.), and optionally replacing with additional CKD subjects who do not have the pre-determined characteristic, to generate a modified set of CKD-Y subjects (e.g., where Y is the pre-determined characteristic). In some embodiments, if the DN vs. CKD-Y score is much higher than the DN vs. CKD score (e.g., >0.1), that is indicative of the kidney damage being a result of the pre-determined characteristic Y.

In some embodiments, the method further comprises analyzing bodily samples of the subject at two or more different time points to yield two or more data sets, and computer processing the two or more data sets to determine the presence, the absence, or the elevated risk of the kidney disease or disorder in the subject. In some embodiments, the method further comprises determining a presence, an absence, or an elevated risk of another type of kidney disease or disorder in the subject, and electronically outputting a report that identifies the presence, the absence, or the elevated risk of the another type of kidney disease or disorder in the subject. In some embodiments, the method further comprises analyzing bodily samples of the subject at two or more different time points to yield two or more data sets, and computer processing the two or more data sets to determine a presence, an absence, or an elevated risk of another type of kidney disease or disorder in the subject.

In another aspect, the present disclosure provides a method for processing or analyzing a bodily sample of a subject, comprising: (a) analyzing the bodily sample to yield a data set comprising one or more levels of gene expression products in the bodily sample, which one or more levels of gene expression products correspond to a set of genes associated with a kidney disease or disorder; (b) computer processing the data set from (a) to determine a presence, an absence, or an elevated risk of the kidney disease or disorder in the subject at a sensitivity of at least about 80%; and (c) electronically outputting a report that identifies the presence, the absence, or the elevated risk of the kidney disease or disorder in the subject determined in (b).

In some embodiments, the bodily sample is selected from the group consisting of: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample. In some embodiments, the bodily sample is the urine sample. In some embodiments, the urine sample is a fresh urine sample. In some embodiments, the urine sample is a frozen sample (e.g., freshly frozen sample). In some embodiments, the urine sample is a preserved urine sample (e.g., stored at room temperature in preservatives to prevent degradation).

In some embodiments, (a) comprises reverse transcribing ribonucleic acid (RNA) molecules obtained or derived from the bodily sample to yield complementary deoxyribonucleic acid (cDNA) molecules, and sequencing at least a portion of the cDNA molecules to yield sequencing reads. In some embodiments, sequencing reads are mapped to a reference sequence (e.g., a reference genome such as a human genome) to yield the data set, wherein the data set comprises counts of gene transcripts. In some embodiments, each count of a transcript is indicative of a gene expression event.

In some embodiments, (a) comprises reverse transcribing ribonucleic acid (RNA) molecules obtained or derived from the bodily sample to yield complementary deoxyribonucleic acid (cDNA) molecules, and assaying at least a portion of the cDNA molecules by Real-time polymerase chain reaction (RT-PCR, also called qPCR) to yield the data set. In some embodiments, the data set comprises cycle threshold (Ct) values, which are inversely proportional to gene expression levels.

In some embodiments, (a) comprises reverse transcribing ribonucleic acid (RNA) molecules obtained or derived from the bodily sample to yield complementary deoxyribonucleic acid (cDNA) molecules, and assaying at least a portion of the cDNA molecules by microarray analysis (e.g. Affymetrix Microarray) to yield the data set. In some embodiments, the data set comprises counts of gene transcripts. In some embodiments, each count is indicative of a gene expression event. In some embodiments, (a) comprises hybridizing ribonucleic acid (RNA) molecules obtained or derived from the bodily sample using specific probe sets, and assaying the hybridized RNA molecules using a Luminex platform to yield the data set. In some embodiments, the data set comprises counts of gene transcripts.

In some embodiments, (a) comprises selectively enriching at least a portion of the cDNA molecules or the RNA molecules for a set of genomic loci associated with the kidney disease or disorder. In some embodiments, (a) comprises amplifying at least a portion of the cDNA molecules or the RNA molecules. In some embodiments, (a) comprises aligning at least a portion of the sequencing reads to a reference sequence. In some embodiments, (a) comprises generating counts of gene transcripts. In some embodiments, the reference sequence is at least a portion of a human reference genome. In some embodiments, the counts of gene transcripts are normalized to generate normalized counts of gene transcripts for downstream differential gene expression analysis. In some embodiments, the normalizing comprises determining CPM (counts per million), TPM (transcripts per kilobase million), or RPKM/FPKM (reads/fragments per kilobase of exon per million reads/fragments mapped).

In some embodiments, the kidney disease or disorder is selected from the group consisting of: early-stage kidney disease, mid-stage kidney disease, late-stage kidney disease, end-stage kidney disease, asymptomatic kidney disease, diabetic nephropathy, hypertensive nephropathy, IgA nephropathy, membranous nephropathy, minimal change disease, focal segmental glomerulosclerosis (FSGS), NSAIDs induced nephrotoxicity, thin basement membrane nephropathy, amyloidosis, ANCA vasculitis related to endocarditis and other infections, cardiorenal syndrome, IgG4 nephropathy, interstitial nephritis, lithium nephrotoxicity, lupus nephritis, multiple myeloma, polycystic kidney disease, pyelonephritis (kidney infection), renal artery stenosis, renal cyst, rheumatoid arthritis-associated renal disease, and kidney stone. In some embodiments, the kidney disease or disorder is diabetic nephropathy. In some embodiments, diabetic nephropathy is early-stage diabetic nephropathy. In some embodiments, the subject is asymptomatic for the diabetic nephropathy.

In some embodiments, (b) comprises using a trained algorithm to process the data set. In some embodiments, the trained algorithm comprises a trained machine learning algorithm. In some embodiments, the trained machine learning algorithm is selected from the group consisting of: a support vector machine (SVM), a naive Bayes classification, a linear regression, a quantile regression, a logistic regression, a nonlinear regression, a random forest, a neural network, an ensemble learning method, a boosting algorithm, an AdaBoost algorithm, a recursive feature elimination algorithm (RFE), and any combination thereof. In some embodiments, the trained machine learning algorithm comprises the recursive feature elimination (RFE) algorithm. In some embodiments, the trained machine learning algorithm is trained with a plurality of training samples comprising a first set of bodily samples from subjects having the type of kidney disease or disorder of interest (e.g. diabetic nephropathy) and a second set of bodily samples from subjects—having no kidney disease or disorder, wherein the first set of bodily samples and the second set of bodily samples are different from the bodily sample of the subject. In some embodiments, the trained machine learning algorithm is trained with a plurality of training samples comprising a first set of bodily samples from subjects having the type of kidney disease or disorder of interest (e.g. diabetic nephropathy) and a second set of bodily samples from subjects having other types of kidney disease or disorder, wherein the first set of bodily samples and the second set of bodily samples are different from the bodily sample of the subject.

In some embodiments, (a) comprises comparing one or more levels of gene expression products to a reference. In some embodiments, the reference corresponds to a set of gene expression products from a first set of bodily samples from subjects having the type of kidney disease or disorder of interest (e.g. diabetic nephropathy) and/or a second set of bodily samples from subjects having no kidney disease or disorder. In some embodiments, the reference corresponds to a set of gene expression products from a first set of bodily samples from subjects having the type of kidney disease or disorder of interest (e.g. diabetic nephropathy) and/or a second set of bodily samples from subjects having other types of kidney disease or disorder.

In some embodiments, the method further comprises detecting the presence or the elevated risk of the kidney disease or disorder in the subject at a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at an Area Under the Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99.

In some embodiments, the method further comprises identifying a clinical intervention for the subject based at least in part on the presence or the elevated risk of the kidney disease or disorder determined in (b). In some embodiments, the clinical intervention is selected from the group consisting of: a drug treatment, intensive glycemic control, high blood pressure control, lower high cholesterol, foster bone health, diet control, lifestyle changes, weight loss, exercise, tobacco cessation, manage alcohol intake, reduce/quit drugs of abuse, and avoiding NSAIDs. In some embodiments, the drug relies on the blockage of the renin-angiotensin aldosterone system (RAAS, e.g., angiotensin-converting enzyme (ACE) inhibitors and angiotensin II receptor blockers (ARBs)). In some embodiments, the drug is an experimental therapy, such as a drug targeting vasculature/hemodynamic effects, a drugs targeting inflammation, and a drug targeting oxidative stress.

In some embodiments, (b) comprises analyzing a first set of genes that differentially distinguishes between a first kidney disease or disorder and negative (NEG) subjects and a second set of genes that differentially distinguishes between the first kidney disease or disorder and a second kidney disease or disorder. In some embodiments, (b) comprises analyzing a first set of genes that differentially distinguishes between diabetic nephropathy (DN) and negative (NEG) subjects and a second set of genes that differentially distinguishes between DN and other chronic kidney diseases (CKD). In some embodiments, the first set of genes is selected from the group of genes listed in Table 3 and Table 5, and wherein the second set of genes is selected from the group of genes listed in Table 4 and Table 6. In some embodiments, (b) comprises generating a first DN vs. NEG score based on the first set of genes and a second DN vs. CKD score based on the second set of genes. In some embodiments, the first DN vs. NEG score is indicative of glomerular injury when greater than 0.5, or is indicative of tubular injury when less than 0.5. In some embodiments, (b) comprises analyzing different male-specific or female-specific sets of genes based on a gender of the subject.

In some embodiments, the method further comprises analyzing bodily samples of the subject at two or more different time points to yield two or more data sets, and computer processing the two or more data sets to determine the presence, the absence, or the elevated risk of the kidney disease or disorder in the subject. In some embodiments, the method further comprises determining a presence, an absence, or an elevated risk of another type of kidney disease or disorder in the subject, and electronically outputting a report that identifies the presence, the absence, or the elevated risk of the another type of kidney disease or disorder in the subject. In some embodiments, the method further comprises analyzing bodily samples of the subject at two or more different time points to yield two or more data sets, and computer processing the two or more data sets to determine a presence, an absence, or an elevated risk of another type of kidney disease or disorder in the subject.

In some embodiments, the sensitivity and specificity of determining the presence of the elevated risk of the kidney disease or disorder in the subject comprises a percentage of independent test samples associated with presence or elevated risk of the kidney disease or disorder that are correctly determined as having the presence or the elevated risk of the kidney disease or disorder.

In some embodiments, the method further comprises determining the presence, the absence, or the elevated risk of the kidney disease or disorder in the subject at a specificity of at least about 80%. In some embodiments, the method further comprises (d) electronically outputting a report that identifies the presence or absence of another type of kidney disease or disorder in the subject.

In another aspect, the present disclosure provides a method for processing or analyzing a bodily sample of a subject, comprising: (a) sequencing ribonucleic acid (RNA) molecules obtained or derived from the bodily sample to yield sequencing reads indicative of counts of gene transcripts in the bodily sample, which counts of gene transcripts correspond to a set of genes associated with a kidney disease or disorder; (b) computer processing the counts of gene transcripts from (a) to determine a presence, an absence, or an elevated risk of the kidney disease or disorder in the subject; and (c) electronically outputting a report that identifies the presence, the absence, or the elevated risk of the kidney disease or disorder in the subject determined in (b).

In some embodiments, the bodily sample is selected from the group consisting of: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample. In some embodiments, the bodily sample is the urine sample. In some embodiments, the urine sample is a fresh urine sample. In some embodiments, the urine sample is a frozen sample (e.g., freshly frozen sample). In some embodiments, the urine sample is a preserved urine sample (e.g., stored at room temperature in preservatives to prevent degradation).

In some embodiments, (a) comprises reverse transcribing ribonucleic acid (RNA) molecules obtained or derived from the bodily sample to yield complementary deoxyribonucleic acid (cDNA) molecules, and sequencing at least a portion of the cDNA molecules to yield sequencing reads. In some embodiments, sequencing reads are mapped to a reference sequence (e.g., a reference genome such as a human genome) to yield the data set, wherein the data set comprises counts of gene transcripts. In some embodiments, each count of a transcript is indicative of a gene expression event. In some embodiments, (a) comprises selectively enriching at least a portion of the cDNA molecules for a set of genomic loci associated with the kidney disease or disorder.

In some embodiments, (a) comprises selectively enriching at least a portion of the cDNA molecules for a set of genomic loci associated with the kidney disease or disorder. In some embodiments, (a) comprises amplifying at least a portion of the cDNA molecules. In some embodiments, (a) comprises aligning at least a portion of the sequencing reads to a reference sequence to generate counts of gene transcripts. In some embodiments, the reference sequence is at least a portion of a human reference genome. In some embodiments, (a) comprises generating counts of gene transcripts. In some embodiments, the reference sequence is at least a portion of a human reference genome. In some embodiments, the counts of gene transcripts are normalized to generate normalized counts of gene transcripts for downstream differential gene expression analysis. In some embodiments, the normalizing comprises determining CPM (counts per million), TPM (transcripts per kilobase million), or RPKM/FPKM (reads/fragments per kilobase of exon per million reads/fragments mapped).

In some embodiments, the kidney disease or disorder is selected from the group consisting of: early-stage kidney disease, mid-stage kidney disease, late-stage kidney disease, end-stage kidney disease, asymptomatic kidney disease, diabetic nephropathy, hypertensive nephropathy, IgA nephropathy, membranous nephropathy, minimal change disease, focal segmental glomerulosclerosis (FSGS), NSAIDs induced nephrotoxicity, thin basement membrane nephropathy, amyloidosis, ANCA vasculitis related to endocarditis and other infections, cardiorenal syndrome, IgG4 nephropathy, interstitial nephritis, lithium nephrotoxicity, lupus nephritis, multiple myeloma, polycystic kidney disease, pyelonephritis (kidney infection), renal artery stenosis, renal cyst, rheumatoid arthritis-associated renal disease, and kidney stone. In some embodiments, the kidney disease or disorder is diabetic nephropathy. In some embodiments, diabetic nephropathy is early-stage diabetic nephropathy. In some embodiments, the subject is asymptomatic for the diabetic nephropathy.

In some embodiments, (b) comprises using a trained algorithm to process the data set. In some embodiments, the trained algorithm comprises a trained machine learning algorithm. In some embodiments, the trained machine learning algorithm is selected from the group consisting of: a support vector machine (SVM), a naive Bayes classification, a linear regression, a quantile regression, a logistic regression, a nonlinear regression, a random forest, a neural network, an ensemble learning method, a boosting algorithm, an AdaBoost algorithm, a recursive feature elimination algorithm (RFE), and any combination thereof. In some embodiments, the trained machine learning algorithm comprises the recursive feature elimination (RFE) algorithm. In some embodiments, the trained machine learning algorithm is trained with a plurality of training samples comprising a first set of bodily samples from subjects having the type of kidney disease or disorder of interest (e.g. diabetic nephropathy) and a second set of bodily samples from subjects having no kidney disease or disorder, wherein the first set of bodily samples and the second set of bodily samples are different from the bodily sample of the subject. In some embodiments, the trained machine learning algorithm is trained with a plurality of training samples comprising a first set of bodily samples from subjects having the type of kidney disease or disorder of interest (e.g. diabetic nephropathy) and a second set of bodily samples from subjects having other types of kidney disease or disorder, wherein the first set of bodily samples and the second set of bodily samples are different from the bodily sample of the subject.

In some embodiments, (a) comprises comparing the counts of gene transcripts (e.g., one or more levels of gene expression products) to a reference. In some embodiments, the reference corresponds to a set of gene expression products from a first set of bodily samples from subjects having the type of kidney disease or disorder of interest (e.g. diabetic nephropathy) and/or a second set of bodily samples from subjects having no kidney disease or disorder. In some embodiments, the reference corresponds to a set of gene expression products from a first set of bodily samples from subjects having the type of kidney disease or disorder of interest (e.g. diabetic nephropathy) and/or a second set of bodily samples from subjects having other types of kidney disease or disorder.

In some embodiments, the method further comprises detecting the presence, the absence, or the elevated risk of the kidney disease or disorder in the subject at a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at an Area Under the Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99.

In some embodiments, the method further comprises identifying a clinical intervention for the subject based at least in part on the presence or the elevated risk of the kidney disease or disorder determined in (b). In some embodiments, the clinical intervention is selected from the group consisting of: a drug treatment, intensive glycemic control, high blood pressure control, lower high cholesterol, foster bone health, diet control, lifestyle changes, weight loss, exercise, tobacco cessation, manage alcohol intake, reduce/quit drugs of abuse, and avoiding NSAIDs. In some embodiments, the drug relies on the blockage of the renin-angiotensin aldosterone system (RAAS, e.g., angiotensin-converting enzyme (ACE) inhibitors and angiotensin II receptor blockers (ARBs)). In some embodiments, the drug is an experimental therapy, such as a drug targeting vasculature/hemodynamic effects, a drugs targeting inflammation, and a drug targeting oxidative stress.

In some embodiments, (b) comprises analyzing a first set of genes that differentially distinguishes between a first kidney disease or disorder and negative (NEG) subjects and a second set of genes that differentially distinguishes between the first kidney disease or disorder and a second kidney disease or disorder. In some embodiments, (b) comprises analyzing a first set of genes that differentially distinguishes between diabetic nephropathy (DN) and negative (NEG) subjects and a second set of genes that differentially distinguishes between DN and other chronic kidney diseases (CKD). In some embodiments, the first set of genes is selected from the group of genes listed in Table 3 and Table 5, and wherein the second set of genes is selected from the group of genes listed in Table 4 and Table 6. In some embodiments, (b) comprises generating a first DN vs. NEG score based on the first set of genes and a second DN vs. CKD score based on the second set of genes. In some embodiments, the first DN vs. NEG score is indicative of glomerular injury when greater than 0.5, or is indicative of tubular injury when less than 0.5. In some embodiments, (b) comprises analyzing different male-specific or female-specific sets of genes based on a gender of the subject.

In some embodiments, the method further comprises analyzing bodily samples of the subject at two or more different time points to yield two or more data sets, and computer processing the two or more data sets to determine the presence, the absence, or the elevated risk of the kidney disease or disorder in the subject. In some embodiments, the method further comprises determining a presence, an absence, or an elevated risk of another type of kidney disease or disorder in the subject, and electronically outputting a report that identifies the presence, the absence, or the elevated risk of the another type of kidney disease or disorder in the subject. In some embodiments, the method further comprises analyzing bodily samples of the subject at two or more different time points to yield two or more data sets, and computer processing the two or more data sets to determine a presence, an absence, or an elevated risk of another type of kidney disease or disorder in the subject.

In another aspect, the present disclosure provides a method for processing or analyzing a bodily sample of a subject, comprising: (a) assaying a plurality of cells obtained or derived from the bodily sample to yield a data set comprising quantitative measures of a set of cell-based biomarkers comprising proteins associated with a kidney disease or disorder in the plurality of cells; (b) computer processing the data set from (a) to determine a presence, an absence, or an elevated risk of the kidney disease or disorder in the subject; and (c) electronically outputting a report that identifies the presence, the absence, or the elevated risk of the kidney disease or disorder in the subject determined in (b). In some embodiments, the method further comprises electronically outputting a report that identifies a presence, an absence, or an elevated risk of other types of kidney disease or disorder in the subject determined in (b).

In some embodiments, the bodily sample is selected from the group consisting of: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample. In some embodiments, the bodily sample is the urine sample. In some embodiments, the urine sample is a fresh urine sample, a frozen urine sample (e.g., freshly frozen urine sample), or a preserved urine sample (e.g., stored at room temperature in preservatives to prevent degradation).

In some embodiments, (a) comprises using a cellular assay selected from the group consisting of: ELISA, flow-cytometry, LC/MS, confocal microscopy. In some embodiments, the set of cell-based biomarkers comprising proteins associated with the kidney disease or disorder comprises at least one protein selected from the group consisting of: proteins encoded by a genomic locus associated with the kidney disease or disorder. In some embodiments, the kidney disease or disorder is selected from the group consisting of: early-stage kidney disease, mid-stage kidney disease, late-stage kidney disease, end-stage kidney disease, asymptomatic kidney disease, diabetic nephropathy, hypertensive nephropathy, IgA nephropathy, membranous nephropathy, minimal change disease, focal segmental glomerulosclerosis (FSGS), NSAIDs induced nephrotoxicity, thin basement membrane nephropathy, amyloidosis, ANCA vasculitis related to endocarditis and other infections, cardiorenal syndrome, IgG4 nephropathy, interstitial nephritis, lithium nephrotoxicity, lupus nephritis, multiple myeloma, polycystic kidney disease, pyelonephritis (kidney infection), renal artery stenosis, renal cyst, rheumatoid arthritis-associated renal disease, and kidney stone. In some embodiments, the kidney disease or disorder is diabetic nephropathy. In some embodiments, diabetic nephropathy is early-stage diabetic nephropathy. In some embodiments, the subject is asymptomatic for the diabetic nephropathy.

In some embodiments, (b) comprises using a trained algorithm to process the data set. In some embodiments, the trained algorithm comprises a trained machine learning algorithm. In some embodiments, the trained machine learning algorithm is selected from the group consisting of: a support vector machine (SVM), a naive Bayes classification, a linear regression, a quantile regression, a logistic regression, a nonlinear regression, a random forest, a neural network, an ensemble learning method, a boosting algorithm, an AdaBoost algorithm, a recursive feature elimination algorithm (RFE), and any combination thereof. In some embodiments, the trained machine learning algorithm comprises the recursive feature elimination algorithm. In some embodiments, the trained machine learning algorithm is trained with a plurality of training samples comprising a first set of bodily samples from subjects having the type of kidney disease or disorder of interest (e.g. diabetic nephropathy) and a second set of bodily samples from subjects having no kidney disease or disorder, wherein the first set of bodily samples and the second set of bodily samples are different from the bodily sample of the subject. In some embodiments, the trained machine learning algorithm is trained with a plurality of training samples comprising a first set of bodily samples from subjects having the type of kidney disease of interest (e.g. diabetic nephropathy) and a second set of bodily samples from subjects having other types of kidney disease or disorder, wherein the first set of bodily samples and the second set of bodily samples are different from the bodily sample of the subject.

In some embodiments, (a) comprises comparing the one or more levels of cell-based biomarkers to a reference. In some embodiments, the reference corresponds to a set of cell-based biomarkers from a first set of bodily samples from subjects having the type of kidney disease or disorder (e.g. diabetic nephropathy) and/or a second set of bodily samples from subjects having no kidney disease or disorder. In some embodiments, the reference corresponds to a set of gene expression products from a first set of bodily samples from subjects having the type of kidney disease or disorder of interest (e.g. diabetic nephropathy) and/or a second set of bodily samples from subjects having other types of kidney disease or disorder, wherein the first set of bodily samples and the second set of bodily samples are different from the bodily sample of the subject.

In some embodiments, the method further comprises detecting the presence or the elevated risk of the kidney disease or disorder in the subject at a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the kidney disease or disorder in the subject at an Area Under the Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99.

In some embodiments, the method further comprises identifying a clinical intervention for the subject based at least in part on the presence or the elevated risk of the kidney disease or disorder determined in (b). In some embodiments, the clinical intervention is selected from the group consisting of: a drug treatment, intensive glycemic control, high blood pressure control, lower high cholesterol, foster bone health, diet control, lifestyle changes, weight loss, exercise, tobacco cessation, manage alcohol intake, reduce/quit drugs of abuse, and avoiding NSAIDs. In some embodiments, the drug relies on the blockage of the renin-angiotensin aldosterone system (RAAS, e.g., angiotensin-converting enzyme (ACE) inhibitors and angiotensin II receptor blockers (ARBs)). In some embodiments, the drug is an experimental therapy, such as a drug targeting vasculature/hemodynamic effects, a drugs targeting inflammation, and a drug targeting oxidative stress.

In some embodiments, (b) comprises analyzing a first set of genes that differentially distinguishes between a first kidney disease or disorder and negative (NEG) subjects and a second set of genes that differentially distinguishes between the first kidney disease or disorder and a second kidney disease or disorder. In some embodiments, (b) comprises analyzing a first set of genes that differentially distinguishes between diabetic nephropathy (DN) and negative (NEG) subjects and a second set of genes that differentially distinguishes between DN and other chronic kidney diseases (CKD). In some embodiments, the first set of genes is selected from the group of genes listed in Table 3 and Table 5, and wherein the second set of genes is selected from the group of genes listed in Table 4 and Table 6. In some embodiments, (b) comprises generating a first DN vs. NEG score based on the first set of genes and a second DN vs. CKD score based on the second set of genes. In some embodiments, the first DN vs. NEG score is indicative of glomerular injury when greater than 0.5, or is indicative of tubular injury when less than 0.5. In some embodiments, (b) comprises analyzing different male-specific or female-specific sets of genes based on a gender of the subject.

In some embodiments, the method further comprises analyzing bodily samples of the subject at two or more different time points to yield two or more data sets, and computer processing the two or more data sets to determine the presence, the absence, or the elevated risk of the kidney disease or disorder in the subject. In some embodiments, the method further comprises determining a presence, an absence, or an elevated risk of another type of kidney disease or disorder in the subject, and electronically outputting a report that identifies the presence, the absence, or the elevated risk of the another type of kidney disease or disorder in the subject. In some embodiments, the method further comprises analyzing bodily samples of the subject at two or more different time points to yield two or more data sets, and computer processing the two or more data sets to determine a presence, an absence, or an elevated risk of another type of kidney disease or disorder in the subject.

In another aspect, the present disclosure provides a method for processing or analyzing a bodily sample of a subject, comprising: (a) sequencing nucleic acid molecules obtained or derived from the bodily sample to yield data comprising one or more quantitative measures of a set of genes associated with a kidney disease or disorder, wherein the sequencing comprises using Illumina RNA prep with enrichment tagmentation kit, Illumina Truseq exome kit, Agilent sureselect target enrichment system, or KAPA RNA HyperPrep kit from Roche; (b) computer processing the data from (a) to determine a presence or an elevated risk of the kidney disease or disorder in the subject; and (c) electronically outputting a report that identifies the presence or the elevated risk of the kidney disease or disorder in the subject determined in (b).

In another aspect, the present disclosure provides a method for processing or analyzing a bodily sample of a subject, comprising: (a) assaying the bodily sample or a derivative thereof to yield data comprising one or more quantitative measures of a set of biomarkers associated with a kidney disease or disorder in the bodily sample or the derivative thereof, wherein the set of biomarkers comprises at least 5 biomarkers selected from the group consisting of: biomarkers listed in Table 3, biomarkers listed in Table 4, biomarkers listed in Table 5, and biomarkers listed in Table 6; (b) computer processing the data from (a) to determine a presence, an absence, or an elevated risk of the kidney disease or disorder in the subject; and (c) electronically outputting a report that identifies the presence, the absence, or the elevated risk of the kidney disease or disorder in the subject determined in (b).

In another aspect, the present disclosure provides a kit for processing or analyzing a bodily sample of a subject, comprising a set of probes for identifying a presence, absence, or relative amount of a set of genomic regions associated with a kidney disease or disorder in the bodily sample or a derivative thereof, wherein the set of biomarkers comprises at least 5 biomarkers selected from the group consisting of: biomarkers listed in Table 3, biomarkers listed in Table 4, biomarkers listed in Table 5, and biomarkers listed in Table 6.

In another aspect, the present disclosure provides a method of diagnosing a kidney disease or disorder of a subject, comprising: (a) assaying a bodily sample of the subject or a derivative thereof to yield data comprising one or more quantitative measures of a set of biomarkers associated with a kidney disease or disorder in the bodily sample or the derivative thereof, wherein the set of biomarkers comprises at least 5 biomarkers selected from the group consisting of: biomarkers listed in Table 3, biomarkers listed in Table 4, biomarkers listed in Table 5, and biomarkers listed in Table 6; and (b) providing a diagnosis of the kidney disease or disorder based on a comparison of the set of biomarkers to a set of reference values.

In another aspect, the present disclosure provides a method for processing or analyzing a urine sample of a subject, comprising (a) sequencing ribonucleic acid (RNA) molecules obtained or derived from the urine sample to yield sequencing reads indicative of counts of gene transcripts in the bodily sample, which counts of gene transcripts correspond to a set of genes associated with diabetic nephropathy; (b) computer processing the counts of gene transcripts from (a) to determine a presence, an absence, or an elevated risk of the diabetic nephropathy in the subject; and (c) electronically outputting a report that identifies the presence, the absence, or the elevated risk of the diabetic nephropathy in the subject determined in (b).

In some embodiments, the bodily sample is selected from the group consisting of: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample. In some embodiments, the bodily sample is the urine sample. In some embodiments, the urine sample is a fresh urine sample. In some embodiments, the urine sample is a frozen sample (e.g., freshly frozen sample). In some embodiments, the urine sample is a preserved urine sample (e.g., stored at room temperature in preservatives to prevent degradation).

In some embodiments, (a) comprises reverse transcribing ribonucleic acid (RNA) molecules obtained or derived from the bodily sample to yield complementary deoxyribonucleic acid (cDNA) molecules, and sequencing at least a portion of the cDNA molecules to yield sequencing reads. In some embodiments, sequencing reads are mapped to a reference sequence (e.g., a reference genome such as a human genome) to yield the data set, wherein the data set comprises counts of gene transcripts. In some embodiments, each count of a transcript is indicative of a gene expression event. In some embodiments, (a) comprises selectively enriching at least a portion of the cDNA molecules for a set of genomic loci associated with the diabetic nephropathy.

In some embodiments, (a) comprises selectively enriching at least a portion of the cDNA molecules for a set of genomic loci associated with the diabetic nephropathy. In some embodiments, (a) comprises amplifying at least a portion of the cDNA molecules. In some embodiments, (a) comprises aligning at least a portion of the sequencing reads to a reference sequence to generate counts of gene transcripts. In some embodiments, the reference sequence is at least a portion of a human reference genome. In some embodiments, (a) comprises generating counts of gene transcripts. In some embodiments, the reference sequence is at least a portion of a human reference genome. In some embodiments, the counts of gene transcripts are normalized to generate normalized counts of gene transcripts for downstream differential gene expression analysis. In some embodiments, the normalizing comprises determining CPM (counts per million), TPM (transcripts per kilobase million), or RPKM/FPKM (reads/fragments per kilobase of exon per million reads/fragments mapped).

In some embodiments, the set of genes associated with the diabetic nephropathy comprises at least one gene selected from the group consisting of: genes listed in Table 3, genes listed in Table 4, genes listed in Table 5, and genes listed in Table 6. In some embodiments, the diabetic nephropathy comprises early-stage diabetic nephropathy, mid-stage diabetic nephropathy, late-stage diabetic nephropathy, end-stage diabetic nephropathy, or asymptomatic diabetic nephropathy.

In some embodiments, (b) comprises using a trained algorithm to process the data set. In some embodiments, the trained algorithm comprises a trained machine learning algorithm. In some embodiments, the trained machine learning algorithm is selected from the group consisting of: a support vector machine (SVM), a naive Bayes classification, a linear regression, a quantile regression, a logistic regression, a nonlinear regression, a random forest, a neural network, an ensemble learning method, a boosting algorithm, an AdaBoost algorithm, a recursive feature elimination algorithm (RFE), and any combination thereof. In some embodiments, the trained machine learning algorithm comprises the recursive feature elimination (RFE) algorithm. In some embodiments, the trained machine learning algorithm is trained with a plurality of training samples comprising a first set of urine samples from subjects having the diabetic nephropathy and a second set of urine samples from subjects having no kidney disease, wherein the first set of urine samples and the second set of urine samples are different from the urine sample of the subject. In some embodiments, the trained machine learning algorithm is trained with a plurality of training samples comprising a first set of urine samples from subjects having the diabetic nephropathy and a second set of urine samples from subjects having other types of kidney diseases, wherein the first set of urine samples and the second set of urine samples are different from the urine sample of the subject.

In some embodiments, (a) comprises comparing the counts of gene transcripts (e.g., one or more levels of gene expression products) to a reference. In some embodiments, the reference corresponds to a set of gene expression products from a first set of urine samples from subjects having the diabetic nephropathy and/or a second set of urine samples from subjects having no kidney disease. In some embodiments, the reference corresponds to a set of gene expression products from a first set of urine samples from subjects having the diabetic nephropathy and/or a second set of urine samples from subjects having other types of kidney diseases.

In some embodiments, the method further comprises detecting the presence or the elevated risk of the diabetic nephropathy in the subject at a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the diabetic nephropathy in the subject at a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the diabetic nephropathy in the subject at a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the diabetic nephropathy in the subject at a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the method further comprises detecting the presence or the elevated risk of the diabetic nephropathy in the subject at an Area Under the Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99.

In some embodiments, the method further comprises identifying a clinical intervention for the subject based at least in part on the presence or the elevated risk of the diabetic nephropathy determined in (b). In some embodiments, the clinical intervention is selected from the group consisting of: a drug treatment, intensive glycemic control, high blood pressure control, lower high cholesterol, foster bone health, diet control, lifestyle changes, weight loss, exercise, tobacco cessation, manage alcohol intake, reduce/quit drugs of abuse, and avoiding NSAIDs. In some embodiments, the drug relies on the blockage of the renin-angiotensin aldosterone system (RAAS, e.g., angiotensin-converting enzyme (ACE) inhibitors and angiotensin II receptor blockers (ARBs)). In some embodiments, the drug is an experimental therapy, such as a drug targeting vasculature/hemodynamic effects, a drugs targeting inflammation, and a drug targeting oxidative stress.

In some embodiments, (b) comprises analyzing a first set of genes that differentially distinguishes between diabetic nephropathy (DN) and negative (NEG) subjects and a second set of genes that differentially distinguishes between DN and a second kidney disease or disorder. In some embodiments, (b) comprises analyzing a first set of genes that differentially distinguishes between DN and negative (NEG) subjects and a second set of genes that differentially distinguishes between DN and other chronic kidney diseases (CKD). In some embodiments, the first set of genes is selected from the group of genes listed in Table 3 and Table 5, and the second set of genes is selected from the group of genes listed in Table 4 and Table 6. In some embodiments, (b) comprises generating a first DN vs. NEG score based on the first set of genes and a second DN vs. CKD score based on the second set of genes. In some embodiments, the first DN vs. NEG score is indicative of glomerular injury when greater than 0.5, or is indicative of tubular injury when less than 0.5. In some embodiments, (b) comprises analyzing different male-specific or female-specific sets of genes based on a gender of the subject.

In some embodiments, the method further comprises analyzing urine samples of the subject at two or more different time points to yield two or more data sets, and computer processing the two or more data sets to determine the presence or the elevated risk of the diabetic nephropathy in the subject. In some embodiments, the method further comprises determining a presence, an absence, or an elevated risk of another type of kidney disease or disorder in the subject, and electronically outputting a report that identifies the presence, the absence, or the elevated risk of the another type of kidney disease or disorder in the subject. In some embodiments, the method further comprises analyzing urine samples of the subject at two or more different time points to yield two or more data sets, and computer processing the two or more data sets to determine the presence, the absence, or the elevated risk of another type of kidney disease or disorder in the subject. In some embodiments, the method further comprises analyzing bodily samples of the subject at two or more different time points to yield two or more data sets, and electronically outputting a report that identifies a presence, an absence, or an elevated risk of another type of kidney disease or disorder in the subject.

In another aspect, the present disclosure provides a method of treating a kidney disease or disorder of a subject, comprising (a) diagnosing the kidney disease or disorder of the subject, according to a method of the present disclosure; and (b) treating the subject for the kidney disease or disorder.

In another aspect, the present disclosure provides a method or a platform of evaluating new drug efficacy for treating kidney disease(s) or disorder of a subject, comprising (a) comparing score changes before and after drug treatment; and (b) computer processing genes expression levels that are targeted by the new drug.

Another aspect of the present disclosure provides a non-transitory computer-readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawings will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a flow-chart for a method 100 of kidney disease or disorder identification in a subject.

FIGS. 2A-2D illustrate examples of dimensionality reduction analysis for diabetic nephropathy (DN) across different age groups (FIG. 2A), different gender groups (FIG. 2B), and different race/ethnicity groups (FIG. 2C), including an illustration of batch effects (FIG. 2D).

FIG. 3 illustrates a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a,” “an,” and “the” include plural references unless the context clearly dictates otherwise. For example, the term “a nucleic acid” includes a plurality of nucleic acids, including mixtures thereof.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the term “target” generally refers to a genomic region within a marker gene or marker region. As used herein, the term “reference” generally refers to a sample obtained or derived from a subject who is diagnosed with kidney disease or disorder (kidney disease or disorder patient) or who has received a negative clinical indication of kidney disease or disorder (e.g., a healthy or control subject without kidney disease or disorder).

As used herein, the terms “locus” or “region” are generally interchangeable and refer to a specific genomic region on the genome represented by chromosome number, start position, and end position.

As used herein, the term “subject,” generally refers to an entity or a medium that has testable or detectable genetic information. A subject can be a person or individual, such as a patient. A subject can be a vertebrate, such as, for example, a mammal. Non-limiting examples of mammals include murines, simians, humans, farm animals, sport animals, and pets.

As used herein, the term “sample” or “biological sample” generally refers to a bodily sample or part(s) of a subject, which is obtained and analyzed to measure or to determine the character of the whole, such as a specimen of tissue, cells, blood, urine, or derivatives thereof.

As used herein, the term “biomarker” generally refers to any substance, structure, or process that can be measured in a subject's body or its products and be used to influence or predict a clinical outcome or disease with or without treatment, select an appropriate treatment (or predict whether treatment would be effective), or monitor a current treatment and potentially change the treatment.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase. Amplification may be performed by polymerase chain reaction (PCR), which is based on using DNA polymerase to synthesize new strands of DNA complementary to the initial template strands.

As used herein, the term “polymerase chain reaction (PCR)” generally refers to a method for increasing the concentration of a segment of a target sequence in a mixture of genomic DNA without cloning or purification. This process for amplifying the target sequence may comprise introducing a large excess of two oligonucleotide primers to the DNA mixture containing the desired target sequence, followed by a precise sequence of thermal cycling in the presence of a DNA polymerase. The two primers may be complementary to their respective strands of the double-stranded target sequence. To perform amplification, the mixture may be denatured and the primers may be annealed to their complementary sequences within the target molecule. Following annealing, the primers may be extended with a polymerase so as to form a new pair of complementary strands. The steps of denaturation, primer annealing, and polymerase extension can be repeated many times (e.g., denaturation, annealing and extension constitute one “cycle”; there can be numerous “cycles”) to obtain a high concentration of an amplified segment of the desired target sequence. The length of the amplified segment of the desired target sequence may be determined by the relative positions of the primers with respect to each other, and therefore, this length is a controllable parameter. By virtue of the repeating aspect of the process, the method is referred to as “polymerase chain reaction” (PCR). Because the desired amplified segments of the target sequence become the predominant sequences (in terms of concentration) in the mixture, they are said to be “PCR amplified” and are “PCR products” or “amplicons.”

As used herein, the term “DNA template” generally refers to the sample DNA that contains the target sequence. At the beginning of the reaction, high temperature is applied to the original double-stranded DNA molecule to separate the strands from each other.

As used herein, the term “primer” generally refers to a short piece of single-stranded DNA that are complementary to the DNA template. The polymerase begins synthesizing new DNA from the end of the primer.

As used herein, the term “AUC” or “AUROC” generally refers to an abbreviation for the area under a Receiver Operating Characteristic (ROC) curve. The ROC curve may be a plot of the true positive rate (TPR) against the false positive rate (FPR) for a plurality of different possible thresholds or cut points of a diagnostic test, thereby illustrating the trade-off between sensitivity and specificity depending on the selected cut point (e.g., any increase in sensitivity is accompanied by a decrease in specificity). The area under an ROC curve (AUC) can be a measure for the accuracy of a diagnostic test (e.g., the larger the area, the more accurate the diagnosis), with an optimal value of 1. In comparison, a random test may have an ROC curve lying on the diagonal with an AUC of 0.5 (e.g., representing a random or worthless test).

The International Society of Nephrology estimates that 850 million people worldwide are affected by kidney disease. Diabetic nephropathy (DN) is a major cause of kidney disease and is the most common cause of end-stage renal disease (ESRD). In addition, DN is linked to higher cardiovascular and all-cause morbidity and mortality, so timely diagnosis and treatment are critical. Diabetic nephropathy is a diabetic kidney disease that generally refers to kidney damage that results from having high blood glucose levels due to diabetes. Diabetic nephropathy progresses slowly. With efficient early treatment, one can slow or even stop the progression of the disease. DN may be associated with measurable biomarkers, such as albuminuria and/or low eGFR, in bodily samples of subjects; however, these biomarkers may be non-specific to DN, and may be attributable to other disease or disorders, such as diabetes, hypertension, IgA nephropathy, membranous nephropathy, lupus nephritis, minimal change disease, rheumatoid arthritis, medicine such as NSAIDs use, smoking, excessive alcohol use, drugs of abuse, obesity, urinary tract infection, kidney stone, benign prostate hyperplasia (BPH), etc. Further, patients with controlled diabetes may also have diabetic nephropathy, whereas poorly controlled diabetic patients may have little kidney damage. Moreover, patients with diabetic nephropathy may also have other type(s) of kidney disease or disorder.

DN is an under-diagnosed and mis-diagnosed disease among patients. For example, DN may be under-diagnosed because diagnostics methods such as renal biopsy can be risky (e.g., with a 1.8% mortality rate), expensive, and time-consuming; therefore, many patients opt out of such diagnostic testing. As another example, DN may be mis-diagnosed because diagnostics methods may lack adequate sensitivity and specificity. For example, a urinary albumin assay may be insensitive and non-specific. As another example, imaging tests such as X-rays and ultrasound diagnostics to check the structure and size of a subject's kidney may be time-consuming and indirect, and other panel tests such as urinary sediment test, urine protein electrophoresis (UPEP), serum protein electrophoresis (SPEP), urine blood test, antinuclear antibody (ANA) test, HBV test, HCV test, HIV test may provide limited information, and therefore are indirect and inefficient. In vitro diagnostic techniques, such as those based on proteomics, genomics, and protein biomarkers, may face challenges in accurately detecting, assessing, and monitoring kidney disease or disorder at high sensitivity, specificity, and accuracy. It may also be difficult to identify early diabetic changes, since the only biomarker typically used is albuminuria; however, albuminuria, especially at low levels, may be confounded with many other factors such as hypertension, smoking, alcohol use, drug abuse, obesity, infection, obstruction, etc. Therefore, many patients may miss the best opportunity for early medical intervention or may receive treatment that is unrelated to the cause of the disease. Receiving such unnecessary and/or ineffective treatment may be expensive, time consuming, and cause delays in providing other effective treatments to patients.

Recognizing the need for improved methods for detecting, assessing, and monitoring kidney disease or disorder (e.g., DN) that are fast, inexpensive, non-invasive, and highly sensitive, specific, and accurate, the present disclosure provides methods, systems, and kits for detecting kidney disease or disorders by processing biological samples (e.g., tissue samples, cell samples, and/or bodily fluid samples) obtained from or derived from a subject. For example, nucleic acids, proteins, or cells of biological samples may be analyzed. Biological samples obtained from subjects may be analyzed to measure a presence, absence, or relative assessment of the kidney disease or disorder. The analysis may be performed at a set of genomic regions, such as kidney disease-associated genes or genomic loci. The subjects may include subjects with kidney disease or disorder (e.g., kidney disease or disorder patients) and subjects without kidney disease or disorder (e.g., normal or healthy controls).

Methods of the present disclosure may present numerous advantages over current methods, including: ease, safety, and non-invasiveness of sample collection from subjects, possibility of repeated assays, the use of urine samples which have kidney cells suitable for analysis, a direct method of analysis of kidney injury, suitability for monitoring disease progression and treatment efficacy, suitability for sample collection in a home setting, the ability to perform testing without detailed medical history of a subject, and the ability to detect early diabetic changes (e.g., for asymptomatic subjects).

Using methods and systems of the present disclosure, kidney disease or disorder can be accurately detected using an assay with high sensitivity and specificity in biological samples (e.g., urine samples). The urine-based assay can apply machine learning algorithm to analyze a set of biomarkers to accurately distinguish kidney disease or disorder samples from control samples across various stages (e.g., early-stage, mid-stage, or late-stage) of kidney disease or disorder. Further, the urine-based assay may offer high specificity, thereby facilitating the non-invasive application of kidney disease or disorder associated biomarkers for treatment monitoring of kidney disease or disorder patients. In addition, the urine-based assay may offer higher sensitivity and specificity than those of renal biopsy, currently considered as the gold standard for definitive diagnosis of kidney diseases.

Processing Biological Samples

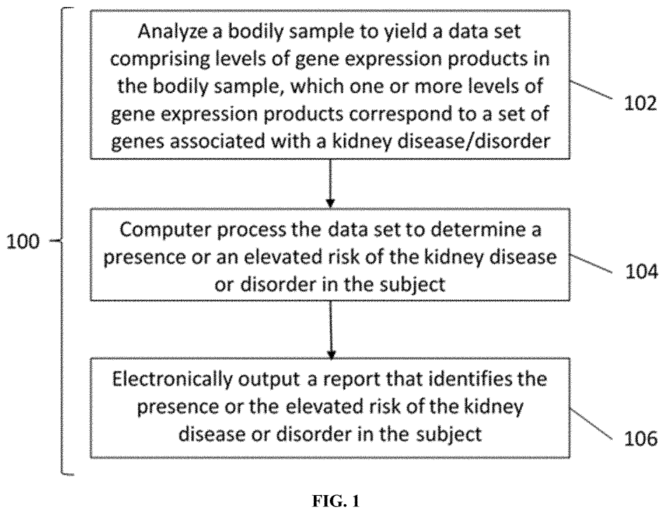

FIG. 1 illustrates a flow-chart for a method 100 of kidney disease or disorder identification in a subject. The method 100 may comprise analyzing a biological sample (e.g., a bodily sample comprising cells, tissue, blood, urine, or derivatives thereof) from a subject (e.g., a patient) to yield a data set comprising levels of gene expression products in the bodily sample (as in operation 102). The levels of gene expression products may correspond to a set of genes associated with a kidney disease or disorder. Next, the method 100 may comprise computer processing the data set to determine a presence or an elevated risk of the kidney disease or disorder in the subject (as in operation 104). Next, the method 100 may comprise electronically outputting a report that identifies the presence or the elevated risk of the kidney disease or disorder in the subject.

The biological sample may be obtained or derived from a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, or a tissue sample from a human subject. The biological sample may be stored in a variety of storage conditions before processing, such as different temperatures (e.g., at room temperature, under refrigeration or freezer conditions, at 4° C., at −18° C., −20° C., or at −80° C., or liquid nitrogen) or different preservatives (e.g., alcohol, formaldehyde, or potassium dichromate, or urine collection and preservation tube from Norgen Biotek Inc.). The biological sample may be a fresh sample (e.g., processed in a suitable time frame to avoid substantial RNA degradation) or a frozen or preserved sample.

The biological sample may be obtained from a subject with a disease or disorder, from a subject that is suspected of having the disease or disorder, or from a subject that does not have or is not suspected of having the disease or disorder. The disease or disorder may be an infectious disease, an immune disorder or disease, a cancer, a genetic disease, a degenerative disease, a lifestyle disease, an injury, a rare disease or an age-related disease. The infectious disease may be caused by bacteria, viruses, fungi, and/or parasites. The disorder or disease may be a kidney disease or disorder. The sample may be taken before and/or after treatment of a subject with a disease or disorder. Samples may be taken before and/or after a treatment. Samples may be taken during a treatment or a treatment regime. Multiple samples may be taken from a subject to monitor the effects of the treatment over time. Multiple samples may be taken from a subject to monitor the disease progression over time. Multiple samples may be taken from a subject to evaluate the possibility of a coexisting kidney disease or disorder. The sample may be taken from a subject known or suspected of having a kidney disease or disorder for which a definitive positive or negative diagnosis is not available via clinical tests.

The sample may be taken from a subject suspected of having a disease or a disorder (e.g., kidney disease or disorder). The sample may be taken from a subject experiencing unexplained symptoms, such as fatigue, nausea, weight loss, aches and pains, weakness, or memory loss. The sample may be taken from a subject having explained symptoms. The sample may be taken from a subject that is asymptomatic for a disease or a disorder (e.g., kidney disease or disorder). The sample may be taken from a subject at risk of developing a disease or disorder (e.g., kidney disease or disorder) due to factors such as familial history, age, environmental exposure, lifestyle risk factors, or presence of other known risk factors.

After obtaining a biological sample from the subject, the biological sample obtained from the subject may be assayed to generate gene expression data indicative of a presence, absence, or relative assessment of a kidney disease or disorder of a subject. For example, a presence, absence, or relative assessment of nucleic acid molecules of the biological sample at a panel of kidney disease or disorder-associated genomic loci (e.g., quantitative measures of gene expression at a plurality of kidney disease or disorder-associated genomic loci) may be indicative of kidney disease or disorder of the subject. The biological samples obtained or derived from the subject may be processed by (i) subjecting the biological sample to conditions that are sufficient to isolate, enrich, or extract a plurality of nucleic acid molecules (e.g., RNA or DNA molecules), and (ii) assaying the plurality of nucleic acid molecules to generate a gene expression profile of the nucleic acid molecules at the panel of kidney disease or disorder-associated genomic loci.

A plurality of nucleic acid molecules may be extracted from the biological sample and subjected to further assaying (e.g., sequencing to generate a plurality of counts of gene transcripts). The nucleic acid molecules may comprise deoxyribonucleic acid (DNA) or ribonucleic acid (RNA). The nucleic acid molecules (e.g., RNA or DNA) may be extracted from the biological sample by a variety of methods, such as a FastDNA Kit protocol from MP Biomedicals or a Allprep DNA/RNAKit from QIAGEN. The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The method may comprise a variety of assays suitable for assessing the presence of gene expression at the kidney disease or disorder-specific markers in a biological sample including Next Generation Sequencing (NGS), Real-time PCR, Microarray analysis and Luminex-based gene expression analysis. The nucleic acid sequencing may be performed by any suitable sequencing methods, such as shotgun sequencing, single-molecule sequencing, nanopore sequencing, semiconductor sequencing, pyrosequencing, sequencing-by-synthesis (SBS), multiplexed PCR based methods, and exome targeted sequencing.

In the workflow of multiplexed PCR, total RNAs may be first reverse transcribed into cDNA. Multiplexed gene specific primers may be hybridized to the target loci (e.g., one or more of the panel of kidney disease or disorder biomarkers or kidney disease or disorder-associated genomic loci), followed by PCR amplification to create amplicons. Primer sequences may be then removed. A second PCR may be performed with sequencing primers to create sequencing-ready fragments. The sequencing may comprise use of commercially available kits and protocols such as Ampliseq by Thermo Fisher or illumina, CleanPlex DNA/RNA amplicon sequencing kit by Paragon Genomics. In the workflow of exome targeted sequencing, total RNAs may be first reverse transcribed into cDNA. A suitable number of rounds of PCR may be performed to sufficiently amplify an initial amount of cDNAs to a desired input quantity. These initially amplified cDNAs may be then hybridized with gene specific probes (e.g., one or more of the panel of kidney disease or disorder biomarkers or kidney disease or disorder-associated gnomic loci). Targeted gene fragments may be selected and enriched. A second PCR may be performed to amplify the enriched products to reach a quantity sufficient for sequencing. The sequencing method may comprise use of commercially available kits and protocols such as Sureselect XT HS2 by Agilent Technologies, Truseq Exome kit by Illumina and HyperPrep kit by Roche.

RNA or DNA molecules may be tagged, e.g., with identifiable tags, to allow for multiplexing of a plurality of samples. Any number of RNA or DNA samples may be multiplexed. For example a multiplexed reaction may contain RNA or DNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial samples. For example, a plurality of samples may be tagged with sample barcodes such that each RNA or DNA molecule may be traced back to the sample (and the subject) from which the RNA or DNA molecule originated. Such tags may be attached to RNA or DNA molecules by ligation or by PCR amplification with primers.

In some embodiments, real-time PCR (RT-PCR) may be used to assess the presence of gene expression at the kidney disease or disorder-specific markers in a biological sample. Only certain target nucleic acids within a population of nucleic acids may be amplified (e.g., one or more of the panel of kidney disease or disorder biomarkers or kidney disease or disorder-associated genomic loci). In some embodiments, up to five gene specific primer/probe sets may be used to selectively amplify certain targets in each well. During amplification, the fluorescent tags carried by the probes may emit fluorescence that can be captured by a camera detector. The intensity level of the fluorescence defined by the cycle threshold (Ct) value may be inversely proportional to the level of gene expression. RTPCR may be performed using any of a number of commercial kits, e.g., provided by Life Technologies, Bio-Rad, Promega, New England Biolabs, etc.

In some embodiments, microarray analysis may be used to assess the presence of gene expression at the kidney disease or disorder-specific markers in a biology sample. Probes for target genes (e.g. one or more of the panel of kidney disease or disorder biomarkers or kidney disease or disorder-associated genomic loci) may be printed in a microarray chip. Total RNAs may be first reverse transcribed into cDNA. Fluorescent dyes may be added during reverse transcription. Labeled cDNA products may be then hybridized with the probes in the microarray chip. After hybridization, the microarray may be dried and scanned by a machine that uses a laser to excite the dye and measures the emission levels with a detector. The amount of fluorescence may be proportional to the levels of gene expression. In some embodiments, Luminex-based gene expression analysis may be used to assess the presence of gene expression at the kidney disease or disorder-specific markers in a biology sample. The assay may be based on direct quantification of the RNA targets for multiplexing of 3 to 80 RNA targets and branched DNA (bDNA) signal amplification technology. The urine sample may be lysed to release the RNAs and incubated overnight with target specific probe sets and Luminex capture beads. Then the signal amplification tree may be built via sequential hybridization of Pre-amplifier, Amplifier, and Label Probe. Each amplification unit may provide a 400× signal amplification, and there may be six amplification units per target RNA copy resulting in a 2,400× signal amplification per copy RNA. The signal may be detected by using the fluorescent reporter molecule, phycoerythrin, on a Luminex instrument for readout and analysis. The Luminex-based gene expression analysis may comprise of using commercially available systems such as QuantiGene Plex Gene Expression Assay by Thermo Fisher.

After subjecting the nucleic acid molecules to sequencing, suitable bioinformatics processes may be performed on the sequence reads to generate the gene expression data indicative of the presence, absence, or relative assessment of the kidney disease or disorder. For example, the sequence reads may be aligned to one or more reference genomes (e.g., a genome of one or more species such as a human genome). The aligned sequence reads may be quantified at a panel of genomic loci to generate the data indicative of a distribution of the presence, absence, or relative assessment of the kidney disease or disorder. For example, quantification of sequences corresponding to a panel of genomic loci associated with kidney disease or disorder may be performed to generate the gene expression data indicative of the presence, absence, or relative assessment of the kidney disease or disorder.

The kidney disease or disorder may be identified or monitored in the subject by using probes configured to selectively enrich nucleic acid (e.g., RNA or DNA) molecules corresponding to the panel of genomic loci (e.g., kidney disease or disorder-associated genomic loci). The probes may be oligonucleotides. The probes may have sequence complementarity with nucleic acid sequences (e.g., about 5 nucleotides, about 10 nucleotides, about 15 nucleotides, about 20 nucleotides, about 25 nucleotides, about 30 nucleotides, about 35 nucleotides, about 40 nucleotides, about 45 nucleotides, about 50 nucleotides, about 55 nucleotides, about 60 nucleotides, about 65 nucleotides, about 70 nucleotides, about 75 nucleotides, about 80 nucleotides, about 85 nucleotides, about 90 nucleotides, about 95 nucleotides, about 100 nucleotides, or more than about 100 nucleotides) from one or more of the individual genomic loci (e.g., kidney disease or disorder-associated genomic loci). The one or more genomic loci (e.g., kidney disease or disorder-associated genomic loci) may comprise 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, or more than about 100 distinct genomic loci (e.g., kidney disease or disorder-associated genomic loci). In some embodiments, the panel of genomic loci comprises one or more kidney disease or disorder-associated genomic loci listed in Table 3, Table 4, Table 5, and/or Table 6.

The biological sample may be processed without any nucleic acid extraction. For example, the processing may comprise assaying the biological sample using probes that are selected for the panel of genomic loci (e.g., kidney disease or disorder-associated genomic loci). The panel of genomic loci (e.g., kidney disease or disorder-associated genomic loci) may comprise 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, or more than about 100 distinct genomic loci (e.g., kidney disease or disorder-associated genomic loci). In some embodiments, the panel of genomic loci comprises one or more kidney disease or disorder-associated genomic loci listed in Table 3, Table 4, Table 5, and/or Table 6.

The processing may comprise assaying the biological sample using probes that are selective for the one or more genomic loci (e.g., kidney disease or disorder-associated genomic loci) among other genomic loci in the biological sample. The probes may be nucleic acid molecules (e.g., RNA or DNA) having sequence complementarity with nucleic acid sequences (e.g., about 5 nucleotides, about 10 nucleotides, about 15 nucleotides, about 20 nucleotides, about 25 nucleotides, about 30 nucleotides, about 35 nucleotides, about 40 nucleotides, about 45 nucleotides, about 50 nucleotides, about 55 nucleotides, about 60 nucleotides, about 65 nucleotides, about 70 nucleotides, about 75 nucleotides, about 80 nucleotides, about 85 nucleotides, about 90 nucleotides, about 95 nucleotides, about 100 nucleotides, or more than about 100 nucleotides) from one or more of the individual genomic loci (e.g., kidney disease or disorder-associated genomic loci). These nucleic acid molecules may be oligonucleotides or enrichment sequences. The assaying of the biological sample using probes that are selected for the one or more genomic loci (e.g., kidney disease or disorder-associated genomic loci) may comprise use of array hybridization, polymerase chain reaction (PCR), or nucleic acid sequencing (e.g., DNA sequencing or RNA sequencing).

The assay readouts may be quantified at one or more of the panel of genomic loci (e.g., kidney disease or disorder-associated genomic loci) to generate the gene expression data indicative of a presence, absence, or relative assessment of the kidney disease or disorder. For example, quantification of array hybridization or polymerase chain reaction (PCR) corresponding to the panel of kidney disease or disorder-associated genomic loci may be performed to generate gene expression data at the panel of kidney disease or disorder-associated genomic loci in the biological sample. Assay readouts may comprise quantitative PCR (qPCR) values, digital PCR (dPCR) values, digital droplet PCR (ddPCR) values, fluorescence values, etc.

Kits

The present disclosure provides kits for identifying or monitoring a kidney disease or disorder in a subject. A kit may comprise probes for identifying a presence, absence, or relative amount of sequences at the panel of kidney disease or disorder-associated genomic loci in a biological sample of the subject, which may be indicative of a kidney disease or disorder. The probes may be selective for the sequences at the panel of kidney disease or disorder-associated genomic loci in the biological sample. A kit may comprise instructions for using the probes to process the biological sample to generate gene expression data at the panel of kidney disease or disorder-associated genomic loci in a biological sample of the subject.

The probes in the kit may be selective for the sequences at the plurality of kidney disease or disorder-associated genomic loci in the biological sample. The probes in the kit may be configured to selectively enrich nucleic acid (e.g., RNA or DNA) molecules corresponding to the panel of kidney disease or disorder-associated genomic loci. The probes in the kit may be—oligonucleotides. The probes in the kit may have sequence complementarity with nucleic acid sequences from one or more of the kidney disease or disorder-associated genomic loci. The one or more genomic loci (e.g., kidney disease or disorder-associated genomic loci) may comprise 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, or more than about 100 distinct genomic loci (e.g., kidney disease or disorder-associated genomic loci). In some embodiments, the one or more genomic loci comprise one or more kidney disease or disorder-associated genomic loci listed in Table 3, Table 4, Table 5, and/or Table 6.

The instructions in the kit may comprise instructions to assay the biological sample using the probes that are selective for the sequences at the panel of kidney disease or disorder-associated genomic loci in the biological sample. The probes may be nucleic acid molecules (e.g., RNA or DNA) having sequence complementarity with nucleic acid sequences (e.g., about 5 nucleotides, about 10 nucleotides, about 15 nucleotides, about 20 nucleotides, about 25 nucleotides, about 30 nucleotides, about 35 nucleotides, about 40 nucleotides, about 45 nucleotides, about 50 nucleotides, about 55 nucleotides, about 60 nucleotides, about 65 nucleotides, about 70 nucleotides, about 75 nucleotides, about 80 nucleotides, about 85 nucleotides, about 90 nucleotides, about 95 nucleotides, about 100 nucleotides, or more than about 100 nucleotides) from one or more of the individual genomic loci (e.g., kidney disease or disorder-associated genomic loci). These nucleic acid molecules may be oligonucleotides or enrichment sequences. The instructions to assay the biological sample may comprise introductions to perform array hybridization, polymerase chain reaction (PCR), or nucleic acid sequencing (e.g., RNA sequencing or DNA sequencing) to process the biological sample to generate gene expression data indicative of a presence, absence, or relative amount of sequences at the panel of kidney disease or disorder-associated genomic loci in the biological sample, which may be indicative of a kidney disease or disorder. The nucleic acid sequencing may be single-molecule (e.g., single-cell RNA-Seq or single-cell DNA-Seq).

The instructions in the kit may comprise instructions to measure and interpret assay readouts, which may be quantified at one or more of the panel of kidney disease or disorder-associated genomic loci to generate the gene expression data indicative of a presence, absence, or relative amount of sequences at the panel of kidney disease or disorder-associated genomic loci in the biological sample. For example, quantification of array hybridization or polymerase chain reaction (PCR) corresponding to the panel of kidney disease or disorder-associated genomic loci may generate gene expression data at the panel of kidney disease or disorder-associated genomic loci in the biological sample. Assay readouts may comprise quantitative PCR (qPCR) values, digital PCR (dPCR) values, digital droplet PCR (ddPCR) values, fluorescence values, etc., normalized values thereof, or ratio values thereof.

Classifiers

After processing the biological sample from the subject, a classifier may be used to process the gene expression data at the panel of kidney disease or disorder-associated genomic loci to classify the biological sample, thereby identifying or assessing a kidney disease or disorder of the subject. In some embodiments, the classifier may be configured to identify the kidney disease or disorder with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The classifier may comprise a supervised machine learning algorithm or an unsupervised machine learning algorithm. The classifier may comprise a classification and regression tree (CART) algorithm. The classifier may comprise, for example, a support vector machine (SVM), a linear regression, a logistic regression, a nonlinear regression, a neural network, an ensemble learning method, a boosting algorithm, an AdaBoost algorithm, a Random Forest, a deep learning algorithm, a naive Bayes classifier, a recursive feature elimination algorithm, or a combination thereof.

The classifier may be configured to accept a plurality of input variables and to produce one or more output values based on the plurality of input variables. The plurality of input variables may comprise data indicative of a presence, absence, or relative amount of sequences or transcripts corresponding to each of the plurality of kidney disease or disorder-associated genomic loci. For example, an input variable may comprise a number of sequences or transcripts corresponding to or aligning to each of the plurality of kidney disease or disorder-associated genomic loci.

The classifier may have one or more possible output values, each comprising one of a fixed number of possible values (e.g., a linear classifier, a logistic regression classifier, etc.) indicating a classification of the biological sample by the classifier. The classifier may comprise a binary classifier, such that each of the one or more output values comprises one of two values (e.g., {0, 1}, {positive, negative}, or {cancerous, non-cancerous}) indicating a classification of the biological sample by the classifier. The classifier may be another type of classifier, such that each of the one or more output values comprises one of more than two values (e.g., {0, 1, 2}, {positive, negative, or indeterminate}, or {diseased, non-diseased, or indeterminate}) indicating a classification of the biological sample by the classifier. The output values may comprise descriptive labels, numerical values, or a combination thereof. Some of the output values may comprise descriptive labels. Such descriptive labels may provide an identification or indication of the disease state of the subject, and may comprise, for example, positive, negative, diseased, non-diseased, or indeterminate. Such descriptive labels may provide an identification of a treatment for the subject's disease state, and may comprise, for example, a therapeutic intervention, a duration of the therapeutic intervention, and/or a dosage of the therapeutic intervention. Such descriptive labels may provide an identification of secondary clinical tests that may be appropriate to perform on the subject, and may comprise, for example, a biopsy, a blood test, a computed tomography (CT) scan, a magnetic resonance imaging (MRI) scan, an ultrasound scan, a chest X-ray, a positron emission tomography (PET) scan, or a PET-CT scan. Such descriptive labels may provide a prognosis of the disease state of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may comprise, for example, an un-normalized probability value of at least 0. Such continuous output values may indicate a prognosis of the disease or disorder state of the subject and may comprise, for example, an indication of an expected or average risk or severity of kidney disease or disorder of the subject. Such continuous output values may indicate a prediction of the course of treatment to treat the disease or disorder state of the subject and may comprise, for example, an indication of an expected duration of efficacy of the course of treatment. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of being diseased. For example, a binary classification of samples may assign an output value of “negative” or 0 if the sample indicates that the subject has less than a 50% probability of being diseased. In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, and about 99%.

As another example, a classification of samples may assign an output value of “positive” or 1 if the sample indicates that the subject has a probability of being diseased of at least 50%, at least about 55%, at least about 60%, at least about 65%, at least about 70%, at least about 75%, at least about 80%, at least about 85%, at least about 90%, at least about 95%, at least about 98%, or at least about 99%. The classification of samples may assign an output value of “positive” or 1 if the sample indicates that the subject has a probability of being diseased of more than 50%, more than 55%, more than 60%, more than 65%, more than 70%, more than 75%, more than 80%, more than 85%, more than 90%, more than 95%, more than 98%, or more than 99%. The classification of samples may assign an output value of “negative” or 0 if the sample indicates that the subject has a probability of being diseased of less than 50%, less than 45%, less than 40%, less than 35%, less than 30%, less than 25%, less than 20%, less than 10%, less than 5%, less than 2%, or less than 1%. The classification of samples may assign an output value of “negative” or 0 if the sample indicates that the subject has a probability of being diseased of no more than 50%, no more than 45%, no more than 40%, no more than 35%, no more than 30%, no more than 25%, no more than 20%, no more than 10%, no more than 5%, no more than 2%, or no more than 1%. The classification of samples may assign an output value of “indeterminate” or 2 if the sample has not been classified as “positive,” “negative,” 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The classifier may be trained with a plurality of independent training samples. Each of the independent training samples may comprise a biological sample from a subject, associated data obtained by processing the biological sample (as described elsewhere herein), and one or more known output values corresponding to the biological sample (e.g., a clinical diagnosis, prognosis, treatment efficacy, or absence of a disease or disorder such as a kidney disease or disorder of the subject). Independent training samples may comprise biological samples and associated data and outputs obtained from a plurality of different subjects. Independent training samples may comprise biological samples and associated data and outputs obtained at a plurality of different time points from the same subject (e.g., before, after, and/or during a course of treatment to treat a disease or disorder of the subject). Independent training samples may be associated with presence of the kidney disease or disorder (e.g., training samples comprising biological samples and associated data and outputs obtained from a plurality of subjects known to have the kidney disease or disorder). Independent training samples may be associated with absence of the kidney disease or disorder (e.g., training samples comprising biological samples and associated data and outputs obtained from a plurality of subjects who are known to not have a previous diagnosis of the kidney disease or disorder, or otherwise who are asymptomatic for the kidney disease or disorder).

The classifier may be trained with at least about 50, at least about 100, at least about 150, at least about 200, at least about 250, at least about 300, at least about 350, at least about 400, at least about 450, or at least about 500 independent training samples. The independent training samples may comprise samples associated with presence of the kidney disease or disorder and/or samples associated with absence of the kidney disease or disorder. The classifier may be trained with no more than 500, no more than 450, no more than 400, no more than 350, no more than 300, no more than 250, no more than 200, no more than 150, no more than 100, or no more than 50 independent training samples associated with presence of the kidney disease or disorder. In some embodiments, the biological sample is independent of samples used to train the classifier.

The classifier may be trained with a first number of independent training samples associated with presence of the kidney disease or disorder and a second number of independent training samples associated with absence of the kidney disease or disorder. The first number of independent training samples associated with presence of the kidney disease or disorder may be no more than the second number of independent training samples associated with absence of the kidney disease or disorder. The first number of independent training samples associated with presence of the kidney disease or disorder may be equal to the second number of independent training samples associated with absence of the kidney disease or disorder. The first number of independent training samples associated with presence of the kidney disease or disorder may be greater than the second number of independent training samples associated with absence of the kidney disease or disorder.

The classifier may be configured to identify the kidney disease or disorder with an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%, for at least about 50, at least about 100, at least about 150, at least about 200, at least about 250, at least about 300, or more than about 300 independent samples. The accuracy of identifying the kidney disease or disorder by the classifier may be calculated as the percentage of independent test samples (e.g., subjects having the kidney disease or disorder, or apparently healthy subjects with negative clinical test results for the kidney disease or disorder) that are correctly identified or classified as having or not having the kidney disease or disorder, respectively.

The classifier may be configured to identify the kidney disease or disorder with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The PPV of identifying the kidney disease or disorder by the classifier may be calculated as the percentage of biological samples identified or classified as having the kidney disease or disorder that correspond to subjects that truly have the kidney disease or disorder. A PPV may also be referred to as a precision.

The classifier may be configured to identify the kidney disease or disorder with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The NPV of identifying the kidney disease or disorder by the classifier may be calculated as the percentage of biological samples identified or classified as not having the kidney disease or disorder that correspond to subjects that truly do not have the kidney disease or disorder.

The classifier may be configured to identify the kidney disease or disorder with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The clinical sensitivity of identifying the kidney disease or disorder by the classifier may be calculated as the percentage of independent test samples associated with presence of the kidney disease or disorder (e.g., subjects known to have the kidney disease or disorder) that are correctly identified or classified as having the kidney disease or disorder. A clinical sensitivity may also be referred to as a recall.

The classifier may be configured to identify the kidney disease or disorder with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The clinical specificity of identifying the kidney disease or disorder by the classifier may be calculated as the percentage of independent test samples associated with absence of the kidney disease or disorder (e.g., apparently healthy subjects with negative clinical test results for the kidney disease or disorder) that are correctly identified or classified as not having the kidney disease or disorder.

The classifier may be configured to identify the kidney disease or disorder with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The AUC may be calculated as an integral of the Receiver Operator Characteristic (ROC) curve (e.g., the area under the ROC curve) associated with the classifier in classifying biological samples as having or not having the kidney disease or disorder.

The classifier may be adjusted or tuned to improve the performance, accuracy, PPV, NPV, clinical sensitivity, clinical specificity, or AUC of identifying the kidney disease or disorder. The classifier may be adjusted or tuned by adjusting parameters of the classifier (e.g., a set of cutoff values used to classify a sample as described elsewhere herein, or weights of a neural network). The classifier may be adjusted or tuned continuously during the training process or after the training process has completed.

After the classifier is initially trained, a subset of the inputs may be identified as most influential or most important to be included for making high-quality classifications. For example, a subset of the plurality of kidney disease or disorder-associated genomic loci may be identified as most influential or most important to be included for making high-quality classifications or identifications of kidney disease or disorder. The plurality of kidney disease or disorder-associated genomic loci or a subset thereof may be ranked based on metrics indicative of each genomic locus's influence or importance toward making high-quality classifications or identifications of kidney disease or disorder. Such metrics may be used to reduce, in some cases significantly, the number of input variables (e.g., predictor variables) that may be used to train the classifier to a desired performance level (e.g., based on a desired minimum accuracy, PPV, NPV, clinical sensitivity, clinical specificity, or AUC). For example, if training the training algorithm with a plurality comprising several dozen or hundreds of input variables in the classifier results in an accuracy of classification of more than 99%, then training the training algorithm instead with only a selected subset of 1, 2, 3, 4, 5, 6, 7, 8, 9, 10, no more than about 10, no more than about 15, no more than about 20, no more than about 25, no more than about 30, no more than about 35, no more than about 40, no more than about 45, no more than about 50, or no more than about 100 such most influential or most important input variables (e.g., marker genes, marker regions, or other genomic loci) among the plurality results in decreased but still acceptable accuracy of classification (e.g., at least about 70%, at least about 75%, at least about 80%, at least about 85%, at least about 90%, at least about 95%, at least about 96%, at least about 97%, or at least about 98%). The subset may be selected by rank-ordering the entire plurality of input variables and selecting a predetermined number (e.g., 1, 2, 3, 4, 5, 6, 7, 8, 9, 10, 11, 12, 13, 14, 15, 16, 17, 18, 19, 20, 21, 22, 23, 24, 25, no more than about 30, no more than about 35, no more than about 40, no more than about 45, no more than about 50, or no more than about 100) of input variables with the best metrics. In some embodiments, the selected subset of the influential or most important input variables comprises one or more genomic loci listed in Table 2.

Identifying or Monitoring a Kidney Disease or Disorder

After using a classifier to process the gene expression data at the panel of kidney disease or disorder-associated genomic loci to classify the biological sample, a quantitative measure indicative of the presence, absence, or relative assessment of the kidney disease or disorder may be determined (e.g., likelihood or probability of kidney disease or disorder), and the kidney disease or disorder may be identified or a progression or regression of the kidney disease or disorder may be monitored in the subject based at least in part on the quantitative measure (e.g., likelihood or probability of kidney disease or disorder).

The kidney disease or disorder may be identified in the subject with an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The accuracy of identifying the kidney disease or disorder by the classifier may be calculated as the percentage of independent test samples (e.g., subjects having the kidney disease or disorder, or apparently healthy subjects with negative clinical test results for the kidney disease or disorder) that are correctly identified or classified as having or not having the kidney disease or disorder, respectively.

The kidney disease or disorder may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The PPV of identifying the kidney disease or disorder by the classifier may be calculated as the percentage of biological samples identified or classified as having the kidney disease or disorder that correspond to subjects that truly have the kidney disease or disorder. A PPV may also be referred to as a precision.

The kidney disease or disorder may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The NPV of identifying the kidney disease or disorder by the classifier may be calculated as the percentage of biological samples identified or classified as not having the kidney disease or disorder that correspond to subjects that truly do not have the kidney disease or disorder.

The kidney disease or disorder may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The clinical sensitivity of identifying the kidney disease or disorder by the classifier may be calculated as the percentage of independent test samples associated with presence of the kidney disease or disorder (e.g., subjects having the kidney disease or disorder) that are correctly identified or classified as having the kidney disease or disorder. A clinical sensitivity may also be referred to as a recall.

The kidney disease or disorder may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The clinical specificity of identifying the kidney disease or disorder by the classifier may be calculated as the percentage of independent test samples associated with absence of the kidney disease or disorder (e.g., apparently healthy subjects with negative clinical test results for the kidney disease or disorder) that are correctly identified or classified as not having the kidney disease or disorder.

After the kidney disease or disorder is identified in a subject, a stage of the kidney disease or disorder (e.g., early stage, mid-stage, or late-stage) may further be identified. The stage of the kidney disease or disorder may be determined based at least in part on the gene expression data at a panel of kidney disease or disorder-associated genomic loci (e.g., quantitative measures of differential gene expressions at the kidney disease or disorder-associated genomic loci). For example, an early-stage kidney disease or disorder may refer to a stage of kidney disease or disorder before clinical symptoms are manifested in the subject. As another example, a late-stage kidney disease or disorder may refer to a stage of kidney disease or disorder for which the subject has high severity of the kidney disease or disorder and/or is suffering severe impairment of kidney function (e.g., in need of dialysis, renal transplantation or tight diabetes management or tight blood pressure control).

Upon identifying the subject as having the kidney disease or disorder, the subject may be provided with a therapeutic intervention (e.g., prescribing an appropriate course of treatment to treat the kidney disease or disorder of the subject). The therapeutic intervention may comprise an effective dose of medication such as ACE inhibitors or ARBs, drugs targeting the vasculature such as Tie-2 activators, sodium-glucose transport protein 2 (SGLT2) inhibitors and glucagon-like peptide 1 (GLP-1) agonists, anti-inflammatory therapies include inflammatory cytokines inhibitors, pentoxifylline, as well as anti-transforming growth factor a/-epiregulin therapies, anti-oxidative stress therapies include nicotinamide adenine dinucleotide phosphate (NADPH) oxidase inhibitors and allopurinol, an effective dose of insulin for diabetes management, a change in diet or exercise regimen, a surgery, tobacco cessation, avoid NSAIDs, or a combination thereof. If the subject is currently being treated for the kidney disease or disorder with a course of treatment, the therapeutic intervention may comprise a subsequent different course of treatment (e.g., to increase treatment efficacy due to inefficacy or non-response of the current course of treatment).

The therapeutic intervention may comprise recommending the subject for a secondary clinical test to confirm a diagnosis of the kidney disease or disorder. This secondary clinical test may comprise a renal biopsy, an imaging test, a blood test, a computed tomography (CT) scan, a magnetic resonance imaging (MM) scan, an ultrasound scan, a chest X-ray, a positron emission tomography (PET) scan, a PET-CT scan, or any combination thereof.

The subject may be treated upon identifying the subject as having the kidney disease or disorder. Treating the subject may comprise administering an appropriate therapeutic intervention to treat the kidney disease or disorder of the subject. The therapeutic intervention may comprise an effective dose of medication, an effective dose of insulin for diabetes management, a change in diet or exercise regimen, a surgery, or a combination thereof. If the subject is currently being treated for the kidney disease or disorder with a course of treatment, the administered therapeutic intervention may comprise a subsequent different course of treatment (e.g., to increase treatment efficacy due to inefficacy or non-response of the current course of treatment).

If the subject is identified as not having the kidney disease or disorder (e.g. diabetic nephropathy) or a different type of kidney disease is suspected to cause more damage, the medical intervention may comprise recommending the subject for a secondary clinical test to determine the cause of the kidney disease. The secondary clinical test may comprise a renal biopsy. If the subject is a heavy smoker, or a heavy alcohol user, or a drug of abuse user, the medical intervention may comprise recommending the subject to quit smoking, drinking or drug of abuse, and have a second test months after substance cessation. If the subject is morbidly obese, the medical intervention may comprise weight management. The subject may have a second test months after weight loss. If the subject has an ongoing kidney infection or urinary tract infection, the medical intervention may comprise recommending having the infection treated first. The subject may have a second test months after the treatment is complete.

Upon identifying the subject as having an elevated risk of developing the kidney disease or disorder (e.g. diabetic nephropathy), the subject may be provided with a therapeutic intervention (e.g., prescribing and/or administering an appropriate course of preventive treatment to protect the kidneys of the subject). The therapeutic intervention may comprise an effective dose of medication such as ACE inhibitors or ARBs, better glucose control, an effective dose of insulin for diabetes management, a change in diet or exercise regimen, better blood pressure control, avoid NSAIDs, weight management, or a combination thereof.

The gene expression data at the panel of kidney disease or disorder-associated genomic loci (e.g., quantitative measures of gene expression at the kidney disease or disorder-associated genomic loci) may be assessed over a duration of time to monitor a patient (e.g., subject who has an elevated risk of developing the kidney disease, or subject who has the kidney disease or disorder or who is being treated for kidney disease or disorder). In such cases, the quantitative measures of gene expression at the kidney disease or disorder-associated genomic loci of the patient may change during the course of intervention or treatment or care. For example, the quantitative measures of gene expression at the kidney disease or disorder-associated genomic loci of a patient whose kidney disease or disorder is regressing due to an effective intervention or treatment may shift toward the gene expression profile or distribution of a healthy subject. Conversely, for example, the quantitative measures of gene expression at the kidney disease or disorder-associated genomic loci of a patient whose kidney disease or disorder is progressing due to an ineffective intervention or treatment (or receiving no intervention or treatment) may shift toward the gene expression profile or distribution of a subject with more advanced stage kidney disease or disorder.

As another example, the quantitative measures of gene expression at the kidney disease or disorder-associated genomic loci of a patient who has an elevated risk of developing the kidney disease or disorder and is regressing due to an effective preventive treatment may shift toward the gene expression profile or distribution of a healthy subject. Conversely, for example, the quantitative measures of gene expression at the kidney disease or disorder-associated genomic loci of a patient who has an elevated risk of developing the kidney disease and is progressing due to an ineffective intervention or treatment (or receiving no intervention or treatment) may shift toward the gene expression profile or distribution of a subject with overt kidney disease or disorder. As another example, the quantitative measures of gene expression at the kidney disease or disorder-associated genomic loci of a patient give a lower score while lab results show no improvement in albuminuria or eGFR may suggest the coexistence or development of another type of chronic kidney disease.

The progression or regression of the kidney disease or disorder in the subject may be monitored by monitoring a course of intervention or treatment for treating the kidney disease or disorder in the subject. The monitoring may comprise assessing the kidney disease or disorder in the subject at two or more time points. The assessing may be based at least on the gene expression data at a panel of kidney disease or disorder-associated genomic loci (e.g., quantitative measures of gene expression at the kidney disease or disorder-associated genomic loci) determined at each of the two or more time points.

A difference in gene expression data at a panel of kidney disease or disorder-associated genomic loci (e.g., quantitative measures of gene expression at the kidney disease or disorder-associated genomic loci) determined between the two or more time points may be indicative of one or more clinical indications, such as (i) a diagnosis of the kidney disease or disorder in the subject, (ii) a prognosis of the kidney disease or disorder in the subject, (iii) a progression of the kidney disease or disorder in the subject, (iv) a regression of the kidney disease or disorder in the subject, (v) an efficacy of the intervention or course of treatment for treating the kidney disease or disorder in the subject, (vi) an inefficacy of the intervention or course of treatment for treating the kidney disease or disorder in the subject, (vii) a possibility of having another type of kidney disease or disorder, (viii) a possibility of another type of co-existing kidney disease being regressing or progressing, and (ix) kidney damage related to tobacco, alcohol, or drug of abuse.

A difference in gene expression data at a panel of kidney disease or disorder-associated genomic loci (e.g., quantitative measures of gene expression at the kidney disease or disorder-associated genomic loci) determined between the two or more time points may be indicative of a diagnosis of the kidney disease or disorder in the subject. For example, if the kidney disease or disorder was not detected in the subject at an earlier time point but was detected in the subject at a later time point, then the difference may be indicative of a diagnosis of the kidney disease or disorder in the subject. A clinical action or decision may be made based on this indication of diagnosis of the kidney disease or disorder in the subject, e.g., prescribing a new therapeutic intervention for the subject.

A difference in gene expression data at a panel of kidney disease or disorder-associated genomic loci (e.g., quantitative measures of gene expression at the kidney disease or disorder-associated genomic loci) determined between the two or more time points may be indicative of a prognosis of the kidney disease or disorder in the subject.

A difference in gene expression data at a panel of kidney disease or disorder-associated genomic loci (e.g., quantitative measures of gene expression at the kidney disease or disorder-associated genomic loci) determined between the two or more time points may be indicative of a regression of the kidney disease or disorder in the subject. For example, if the kidney disease or disorder was detected in the subject both at an earlier time point and at a later time point, and if the difference is a negative difference (e.g., the presence, absence, or relative assessment of gene expression at a panel of kidney disease or disorder-associated genomic loci (e.g., quantitative measures of gene expression at the kidney disease or disorder-associated genomic loci) decreased from the earlier time point to the later time point), then the difference may be indicative of a regression (e.g., decreased tumor load, tumor burden, or tumor size) of the kidney disease or disorder in the subject. A clinical action or decision may be made based on this indication of the regression, e.g., continuing or ending a current therapeutic intervention for the subject.

A difference in gene expression data at a panel of kidney disease or disorder-associated genomic loci (e.g., quantitative measures of gene expression at the kidney disease or disorder-associated genomic loci) determined between the two or more time points may be indicative of an efficacy of the course of treatment for treating the kidney disease or disorder in the subject. For example, if the kidney disease or disorder was detected in the subject at an earlier time point but was not detected in the subject at a later time point, then the difference may be indicative of an efficacy of the course of treatment for treating the kidney disease or disorder in the subject. A clinical action or decision may be made based on this indication of the efficacy of the course of treatment for treating the kidney disease or disorder in the subject, e.g., continuing or ending a current therapeutic intervention for the subject.

A difference in gene expression data at a panel of kidney disease or disorder-associated genomic loci (e.g., quantitative measures of gene expression at the kidney disease or disorder-associated genomic loci) determined between the two or more time points may be indicative of an inefficacy of the course of treatment for treating the kidney disease or disorder in the subject. For example, if the kidney disease or disorder was detected in the subject both at an earlier time point and at a later time point, and if the difference is a positive or zero difference (e.g., the presence, absence, or relative assessment of gene expression at a panel of kidney disease or disorder-associated genomic loci (e.g., quantitative measures of gene expression at the kidney disease or disorder-associated genomic loci) increased or remained at a constant level from the earlier time point to the later time point), then the difference may be indicative of an inefficacy of the course of treatment for treating the kidney disease or disorder in the subject. A clinical action or decision may be made based on this indication of the inefficacy of the course of treatment for treating the kidney disease or disorder in the subject, e.g., ending a current therapeutic intervention and/or switching to (e.g., prescribing) a different new therapeutic intervention for the subject.

Outputting a report of the kidney disease or disorder

After the kidney disease or disorder is identified or a progression or regression of the kidney disease or disorder is monitored in the subject, a report may be electronically outputted that identifies or provides an indication of the identification, prognosis, regression, elevated risk of the kidney disease or disorder, or the possibility of having another type of kidney disease or disorder in the subject. The subject may not display a kidney disease or disorder (e.g., is asymptomatic of the kidney disease or disorder). The report may be presented on a graphical user interface (GUI) of an electronic device of a user. The user may be the subject, a caretaker, a physician, a nurse, or another health care worker.

The report may include one or more clinical indications such as (i) a diagnosis of the kidney disease or disorder in the subject, (ii) a prognosis of the kidney disease or disorder in the subject, (iii) a progression of the kidney disease or disorder in the subject, (iv) a regression of the kidney disease or disorder in the subject, (v) an efficacy of the intervention or course of treatment for treating the kidney disease or disorder in the subject, (vi) an inefficacy of the intervention or course of treatment for treating the kidney disease or disorder in the subject, (vii) a possibility of having another type of kidney disease or disorder, (viii) a possibility of another type of co-existing kidney disease being regressing or progressing, and (ix) kidney damage related to tobacco, alcohol, or drug of abuse. The report may include one or more clinical actions or decisions made based on these one or more clinical indications.

For example, a clinical indication of a diagnosis of the kidney disease or disorder in the subject may be accompanied with a clinical action of prescribing a new therapeutic intervention for the subject. As another example, a clinical indication of a progression of the kidney disease or disorder in the subject may be accompanied with a clinical action of prescribing a new therapeutic intervention or switching therapeutic interventions (e.g., ending a current treatment and prescribing a new treatment) for the subject. As another example, a clinical indication of a regression of the kidney disease or disorder in the subject may be accompanied with a clinical action of continuing or ending a current therapeutic intervention for the subject. As another example, a clinical indication of an efficacy of the course of treatment for treating the kidney disease or disorder in the subject may be accompanied with a clinical action of continuing or ending a current therapeutic intervention for the subject. As another example, a clinical indication of an inefficacy of the course of treatment for treating the kidney disease or disorder in the subject may be accompanied with a clinical action of ending a current therapeutic intervention and/or switching to (e.g., prescribing) a different new therapeutic intervention for the subject.

Computer Systems

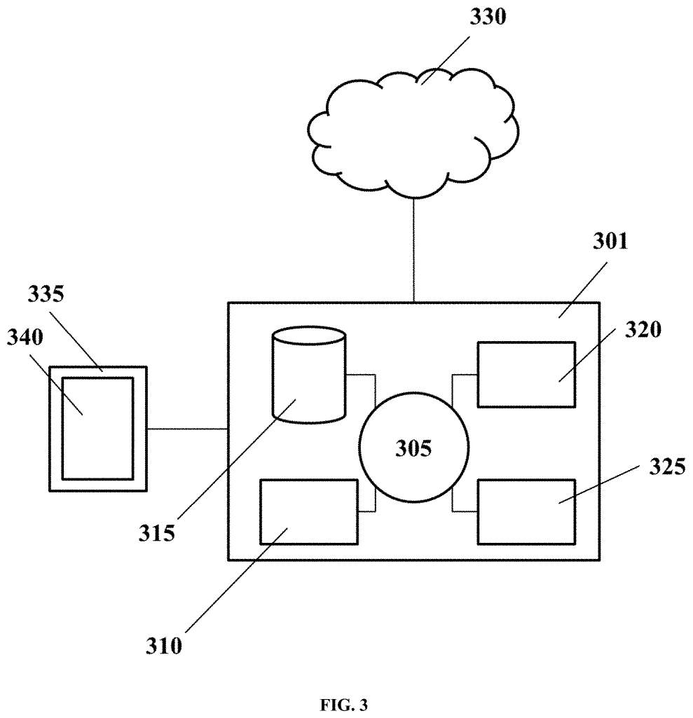

The present disclosure provides computer systems that are programmed to implement methods of the disclosure. FIG. 3 shows a computer system 301 that is programmed or otherwise configured to, for example, determine quantitative measures of gene expression to generate gene expression profiles of RNA molecules at genomic regions; determine a quantitative measure indicative of a presence, absence, elevated risk, or relative assessment of a kidney disease or disorder of a subject; analyze gene expression data; identify or provide an indication of the kidney disease or disorder of the subject; and electronically output a report that identifies or provides an indication of the kidney disease or disorder of the subject.

The computer system 301 can regulate various aspects of analysis, calculation, and generation of the present disclosure, such as, for example, determining quantitative measures of gene expression to generate gene expression profiles of RNA molecules at genomic regions; determining a quantitative measure indicative of a presence, absence, elevated risk, or relative assessment of a kidney disease or disorder of a subject; analyzing gene expression data; identifying or providing an indication of the kidney disease or disorder of the subject; and electronically outputting a report that identifies or provides an indication of the kidney disease or disorder of the subject. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320, and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet.

The network 330 in some cases is a telecommunication and/or data network. The network 330 can include one or more computer servers, which can enable distributed computing, such as cloud computing. For example, one or more computer servers may enable cloud computing over the network 330 (“the cloud”) to perform various aspects of analysis, calculation, and generation of the present disclosure, such as, for example, determining quantitative measures of gene expression to generate gene expression profiles of RNA molecules at genomic regions; determining a quantitative measure indicative of a presence, absence, elevated risk, or relative assessment of a kidney disease or disorder of a subject; analyzing gene expression data; identifying or providing an indication of the kidney disease or disorder of the subject; and electronically outputting a report that identifies or provides an indication of the kidney disease or disorder of the subject. Such cloud computing may be provided by cloud computing platforms such as, for example, Amazon Web Services (AWS), Microsoft Azure, Google Cloud Platform, and IBM cloud. The network 330, in some cases with the aid of the computer system 301, can implement a peer-to-peer network, which may enable devices coupled to the computer system 301 to behave as a client or a server.

The CPU 305 may comprise one or more computer processors and/or one or more graphics processing units (GPUs). The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine-readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 301 can include or be in communication with an electronic display 335 that comprises a user interface (UI) 340 for providing, for example, a visual display of data (e.g., gene expression data) indicative of a presence, absence, or relative assessment of kidney disease or disorder of a subject; a determined presence, absence, elevated risk, or relative assessment of kidney disease or disorder of a subject, an identification of a subject as having kidney disease or disorder; or an electronic report that identifies or provides an indication of the kidney disease or disorder of the subject. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, determine quantitative measures of gene expression to generate gene expression profiles of RNA molecules at genomic regions; determine a quantitative measure indicative of a presence, absence, elevated risk, or relative assessment of a kidney disease or disorder of a subject; analyze gene expression data; identify or provide an indication of the kidney disease or disorder of the subject; and electronically output a report that identifies or provides an indication of the kidney disease or disorder of the subject.

### EXAMPLES

### Example 1

Using methods and systems of the present disclosure, urine samples were analyzed from subjects to non-invasively assess diabetic nephropathy (DN). First, fresh urine specimens were collected from a set of more than 300 subjects using urine collection and preservation cups (120 cc from Norgen Biotek Corp.). The set of subjects included male and female subjects, including those with diabetic nephropathy (DN), those with no diabetes or diabetes without kidney manifestation (NEG), and those with non-diabetic chronic kidney disease patients (CKD).

Next, medical records for the subjects were reviewed, including doctor's notes and lab results were carefully reviewed. Strict selection criteria were applied to select the most representative diabetic nephropathy patients (DN), negative controls (NEG) with no diabetes or diabetes without kidney manifestation, and non-diabetic chronic kidney disease patients (CKD). Next, total RNA samples were isolated from the urine samples and subjected to whole transcriptome RNA sequencing. The library prep kits may be, for example, chosen from Illumina's Nextera RNA Enrichment Tagmentation kit; Illumina's Truseq RNA Exome kit; Agilent's SureSelect XT HS2 RNA prep kit; and KAPA RNA HyperPrep from Roche.

Next, data analysis was performed on the sequencing reads, including dimension reduction via principal component analysis (PCA) analysis, using various parameters such as gender, ethnicity, age, batch effect, etc. Gender was the only parameter observed to form two distinct clusters; therefore, the data set was divided into male and female groups.

Next, to determine the gene signatures related to diabetic nephropathy, two comparisons were performed: DN vs. CKD, and DN vs. NEG. The DN samples used were the same in both comparisons. In each comparison, the DESeq2 library package in R studio was performed to generate a differentially expressed gene list. Alternatively, all genes in our data set (˜13,000 after filtering) were also used.

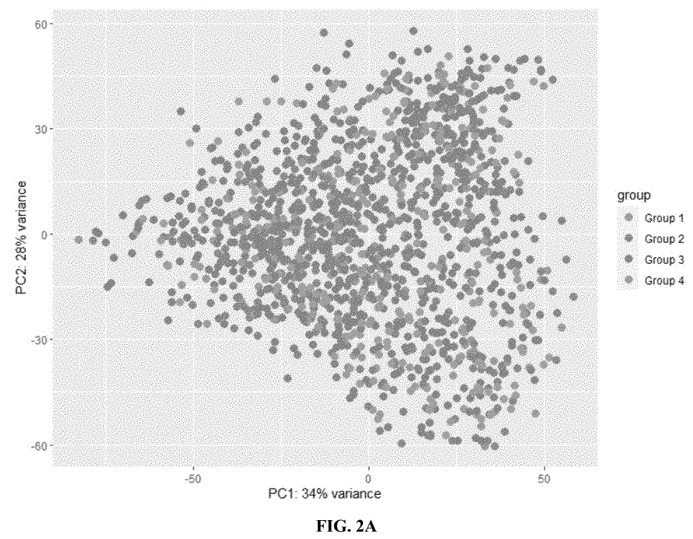

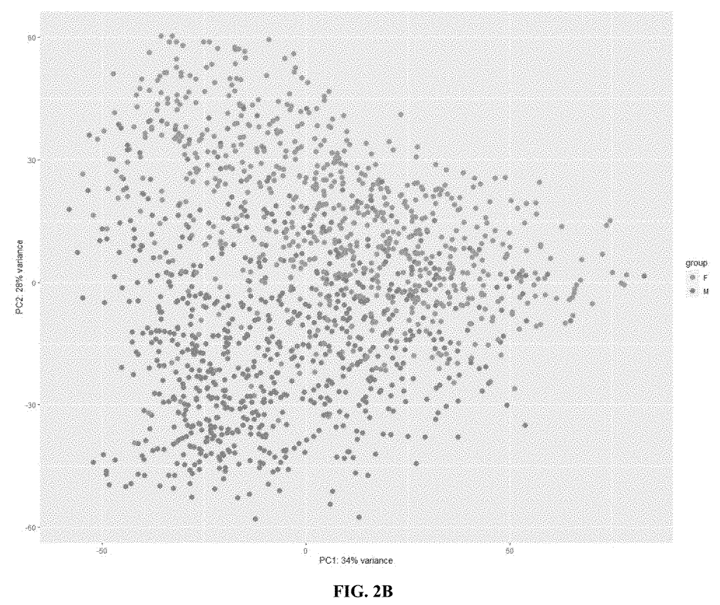

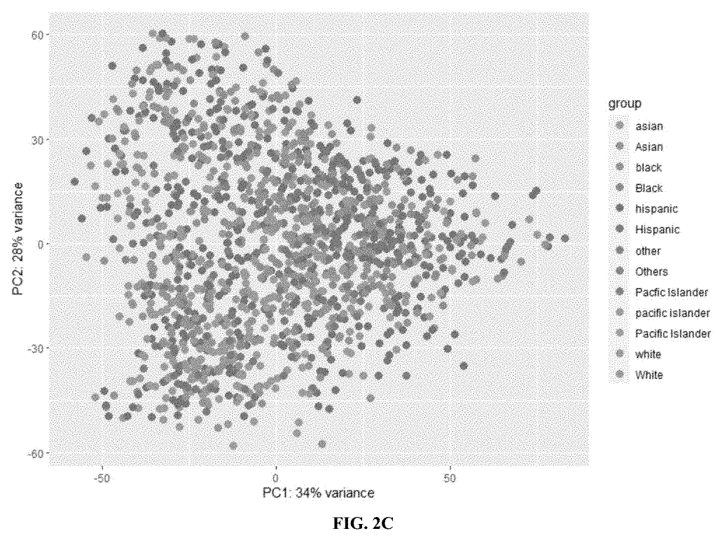

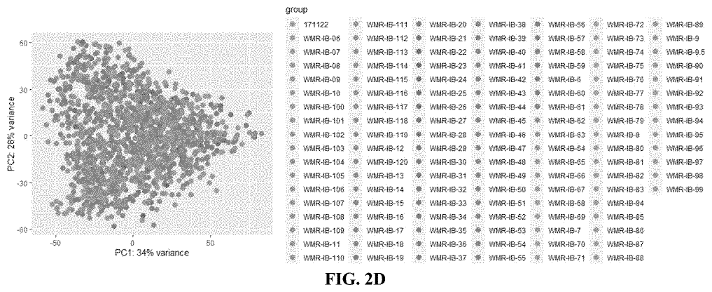

FIGS. 2A-2C illustrate examples of dimensionality reduction analysis for diabetic nephropathy (DN) across different age groups (FIG. 2A), different gender groups (FIG. 2B), and different race/ethnicity groups (FIG. 2C). These figures show that no distinct clusters are formed across different age groups (FIG. 2A), and different race/ethnicity groups (FIG. 2C). Male and female subjects formed two distinct clusters (FIG. 2B), so the data were split into male and female groups for separate analysis.

Next, feature selection in Python was then tested using various classifiers on the patients and the corresponding gene lists. In each test, 80% of the samples were randomly chosen as a training data set, and the remaining 20% of the sample was used as a test data set.

Among the classifiers tested, the Recursive Feature Elimination classifier was determined to yield the best predictive scores. Next, a scoring system was generated in Python using a trained logistic regression classifier, which outputs a probability score (between 0 and 1) that describes the probability of the sample being of a certain group (e.g., has or does not have diabetic nephropathy). If the probability score is above the threshold (e.g., 0.5), then the sample is classified as “yes” or “positive” for DN (e.g., the subject has DN); otherwise, the sample is classified as “no” or “negative” (e.g., the subject does not have DN).

Diabetic nephropathy was typically found to be confidently called only when scores from both the DN vs. CKD model and the DN vs. NEG model were above a pre-determined threshold (e.g., about 0.70). For example, when considering only a single biomarker score, a score of DN vs. NEG that is greater than 0.5 may indicate glomerular injury, while a score of DN vs. NEG that is less than 0.5 may indicate tubular injury. Therefore, performing such a two-biomarker assessment approach advantageously yielded significantly increased specificity, as shown in Table 1.

TABLE 1
Clinical explanations of DN, NEG, and CKD scores in patients

| DN vs. NEG | | DN vs. CKD | | |
|---|---|---|---|---|
| Score | | Score | | |
| No | Yes | No | Yes | Explanation |
| 0.01 | 0.99 | 0.01 | 0.99 | All kidney damage is from diabetes |
| 0.01 | 0.99 | 0.40 | 0.60 | There is a kidney damage. There may be |
| | | | | diabetic nephropathy and a coexisting non- |
| | | | | diabetic kidney disease or disorder. |
| | | | | Overall kidney damage comes more from |
| | | | | diabetes. |
| 0.01 | 0.99 | 0.60 | 0.40 | There is a kidney damage. There may be |
| | | | | diabetic nephropathy and a coexisting |
| | | | | non-diabetic kidney disease or disorder. |
| | | | | Overall kidney damage comes less from |
| | | | | diabetes. |
| 0.01 | 0.99 | 0.99 | 0.01 | There is a kidney damage. |
| | | | | 1. There is no diabetic nephropathy. |
| | | | | Kidney damage comes all from non-diabetic |
| | | | | kidney disease or disorder; |
| | | | | 2. There may be diabetic nephropathy and |
| | | | | a coexisting non-diabetic kidney disease |
| | | | | or disorder. Overall kidney damage comes |
| | | | | predominantly from non-diabetic kidney |
| | | | | disease. |
| 0.50 | 0.50 | 0.01 | 0.99 | Diabetic nephropathy and non-diabetic |
| | | | | kidney disease or disorder coexist. |
| | | | | There may be a non-kidney related |
| | | | | injury that can cause albuminuria. |
| 0.99 | 0.01 | 0.01 | 0.99 | Gene expression pattern for diabetic |
| | | | | nephropathy is formed but the actual |
| | | | | kidney damage comes from another |
| | | | | source. An alternative explanation is |
| | | | | that albuminuria is not from kidney |
| | | | | damage. |
| 0.50 | 0.50 | 0.50 | 0.50 | 1. Diabetic nephropathy may exist and a |
| | | | | non-diabetic kidney disease may coexist; |
| | | | | 2. DN is recovering from effective treatment. |
| 0.60 | 0.40 | 0.60 | 0.40 | 1. Diabetic nephropathy may exist but a |
| | | | | non-diabetic kidney disease may coexist. |
| | | | | Overall kidney damage comes less from |
| | | | | diabetes; |
| | | | | 2. DN is recovering from effective |
| | | | | treatment. |
| 0.40 | 0.60 | 0.40 | 0.60 | 1. Diabetic nephropathy may exist but a |
| | | | | non-diabetic kidney disease may coexist. |
| | | | | Overall kidney damage comes more from |
| | | | | diabetes; |
| | | | | 2. DN is recovering from effective |
| | | | | treatment. |
| 0.99 | 0.01 | 0.99 | 0.01 | 1. There is no diabetic nephropathy; |
| | | | | 2. Albuminuria may come from a non-kidney |
| | | | | related source. |
| | | | | 3. There may be diabetic nephropathy and |
| | | | | a coexisting non-diabetic kidney disease |
| | | | | or disorder. Overall kidney damage comes |
| | | | | predominantly from non-diabetic kidney |
| | | | | disease. |
| | | | | 4. Diabetic nephropathy cannot be detected |
| | | | | after effective treatment. |

Some patients at the time of sample collection had no apparent kidney injury (e.g., they had normal albuminuria and eGFR levels), but then developed microalbuminuria shortly afterwards. These subjects' score patterns were observed to be close to that of DN. Based on their medical history, which may include the existence of other diabetic complications, the model scores were used to reasonably predict that these patients were at high risk of developing DN. Table 2 shows clinical explanations for subjects, including typical cases for predicting diabetic changes in patients with still normal albuminuria.

TABLE 2
Clinical explanations of DN, NEG, and CKD scores in patients

| DN vs. NEG | | DN vs. CKD | | |
|---|---|---|---|---|
| Score | | Score | | |
| No | Yes | No | Yes | Clinical Explanation |
| 0.99 | 0.01 | 0.99 | 0.01 | No kidney damage |
| 0.99 | 0.01 | 0.50 | 0.50 | Gene expressions related to diabetic |
| | | | | changes move towards a DN pattern but |
| | | | | there is no actual kidney damage |
| 0.99 | 0.01 | 0.01 | 0.99 | Gene expressions related to diabetic |
| | | | | changes have reached a DN pattern but |
| | | | | there is no actual kidney damage |
| 0.50 | 0.50 | 0.01 | 0.99 | Gene expressions related to diabetic |
| | | | | changes have reached a DN pattern. |
| | | | | There may be very mild kidney injury |
| | | | | that cannot be detected by urinary |
| | | | | albumin test. |
| 0.01 | 0.99 | 0.01 | 0.99 | Gene expressions related to diabetic |
| | | | | changes have reached a DN pattern. |
| | | | | There may be very mild kidney injury |
| | | | | that cannot be detected by urinary |
| | | | | albumin test. It is possible that |
| | | | | albuminuria/creatinine ratio will be >30 |
| | | | | mg/g in months if left untreated. |

If a patient is found to have higher risk of developing DN, a therapeutic intervention may be prescribed and/or administered to the patient, such as near-normal blood glucose control, antihypertensive treatment, and restriction of dietary proteins. Various drugs may be administered, such as hormones (e.g., insulin), sulfonylureas, biguanides, angiotensin-converting enzyme (ACE) inhibitors, angiotensin receptor blockers (ARBs), beta-adrenergic blocking agents, calcium channel blockers, and diuretics. However, it is actually more important to rule out or exclude patients who do not have diabetic nephropathy, but who present with confounding symptoms or biomarkers, so that effective treatment can be focused on other causes at an earlier time point, thereby increasing efficiency, decreasing costs and side effects.

Further, the biopsy-confirmed samples were scored, and most of them matched. In each comparison, whether DN vs. CKD or DN vs. NEG, several thousand random split events were performed (e.g., 2,000 to 5,000 times). The most frequently occurring genes were identified and selected as the gene signature for diabetic nephropathy. In this case, four sets of gene signatures were identified: two for male and two for female (e.g., DN vs. CKD and DN vs. NEG for each), as shown below in Table 3, Table 4, Table 5, and Table 6. For each of these 4 tables, four or five sets (of various sizes) of strongly predictive differential gene expression markers are provided.

TABLE 3
Differential gene expression markers for assessment of DN vs. NEG (female subjects)

| Number of Genes | 55 | 65 | 81 | 89 | 132 |
|---|---|---|---|---|---|
| Percentage of | 94.62% | 97.18% | 98.21% | 99.23% | 100% |
| Positive Samples | | | | | |
| with a Score | | | | | |
| of >0.90 and | | | | | |
| Negative Samples | | | | | |
| with a Score | | | | | |
| of <0.1 | | | | | |
| Gene List | RPL22 | RPL22 | LOC100133331 | LOC100133331 | LOC100133331 |
| | SNORA61 | SNORA61 | RPL22 | RPL22 | RPL22 |
| | VCAM1 | VCAM1 | MST1L | SNORA61 | MST1L |
| | NES | NES | SNORA61 | VCAM1 | XKR8 |
| | PAPPA2 | PAPPA2 | VCAM1 | NES | SNORA61 |
| | NPHS2 | NPHS2 | NES | PAPPA2 | VCAM1 |
| | IGFN1 | IGFN1 | PAPPA2 | NPHS2 | NES |
| | CHI3L1 | CHI3L1 | NPHS2 | IGFN1 | PAPPA2 |
| | PIGR | PIGR | IGFN1 | CHI3L1 | NPHS2 |
| | RYR2 | RYR2 | CHI3L1 | CHIT1 | IGFN1 |
| | ITIH5 | ITIH5 | CHIT1 | PIGR | CHI3L1 |
| | HTRA1 | HTRA1 | PIGR | RYR2 | CHIT1 |
| | HBB | HBB | RYR2 | ITIH5 | PIGR |
| | SNORA45B | SNORA45B | ITIH5 | ANKRD1 | RYR2 |
| | WT1 | WT1 | ANKRD1 | HTRA1 | ITIH5 |
| | SNORD67 | SNORD67 | CYP17A1 | HBB | LIPA |
| | GLYATL1 | GLYAT | HTRA1 | SNORA45B | ANKRD1 |
| | UPK2 | GLYATL1 | HBB | WT1 | CYP17A1 |
| | C3AR1 | UPK2 | SNORA45B | C11orf96 | HTRA1 |
| | PTPRQ | MCAM | WT1 | SNORD67 | HBB |
| | ATP12A | C3AR1 | C11orf96 | GLYAT | SNORA45B |
| | ERICH6B | PTPRQ | SNORD67 | GLYATL1 | PAX6 |
| | DHRS2 | ATP12A | GLYAT | UPK2 | WT1 |
| | IFI27 | ERICH6B | GLYATL1 | MCAM | C11orf96 |
| | SLC12A1 | DHRS2 | UPK2 | C3AR1 | SNORD67 |
| | TPM1 | IFI27 | MCAM | PTPRO | GLYAT |
| | HBA1 | SLC12A1 | C3AR1 | PTPRQ | GLYATL1 |
| | UMOD | TPM1 | PTPRO | ATP12A | MS4A4A |
| | SLC4A1 | GOLGA6L5P | PTPRQ | STARD13 | HEPHL1 |
| | LRRC37A2 | HBA1 | ATP12A | FREM2 | UPK2 |
| | WDR87 | UMOD | FREM2 | ERICH6B | RPS25 |
| | APOC1 | TMEM97 | ERICH6B | DHRS2 | MCAM |
| | ZNF331 | SLC4A1 | DHRS2 | IFI27 | C3AR1 |
| | NR4A2 | LRRC37A2 | IFI27 | SLC12A1 | PTPRO |
| | SERPINE2 | ZNF69 | SLC12A1 | UNC13C | LLPH |
| | UGT1A9 | WDR87 | TPM1 | TPM1 | PTPRQ |
| | AGXT | APOC1 | GOLGA6L5P | IMP3 | ATP12A |
| | MYL9 | ZNF331 | ARRDC4 | GOLGA6L5P | STARD13 |
| | TGM2 | NR4A2 | HBA2 | ARRDC4 | FREM2 |
| | MIOX | PLA2R1 | HBA1 | HBA2 | ERICH6B |
| | IGFBP7 | SERPINE2 | UMOD | HBA1 | DHRS2 |
| | ENPEP | UGT1A9 | LOC388282 | UMOD | IFI27 |
| | SLC1A3 | AGXT | TMEM97 | FAM157C | SLC12A1 |
| | BHMT | THBD | SLC4A1 | NOS2 | UNC13C |
| | DPYSL3 | MYL9 | LRRC37A2 | TMEM97 | TPM1 |
| | SYNPO | TGM2 | ZNF69 | SLC4A1 | IMP3 |
| | SPARC | ABCG1 | WDR87 | LRRC37A2 | GOLGA6L5P |
| | HLA-DRB5 | MIOX | APOC1 | ZNF69 | ARRDC4 |
| | CLIC5 | ENPEP | ZNF331 | WDR87 | HBA2 |
| | RELN | SLC1A3 | NR4A2 | APOC1 | HBA1 |
| | PODXL | F2R | COL3A1 | SULT1C2 | UMOD |
| | FGFR1 | BHMT | SERPINE2 | NR4A2 | ZNF668 |
| | SULF1 | DPYSL3 | UGT1A9 | PLA2R1 | LOC388282 |
| | GPT | SYNPO | AGXT | COL3A1 | PDXDC2P |
| | NR4A3 | SPARC | THBD | HSPE1 | FAM157C |
| | | CLIC5 | MYL9 | SERPINE2 | NOS2 |
| | | TPBG | TGM2 | UGT1A9 | TMEM97 |
| | | RP9 | ANKRD20A11 | AGXT | IKZF3 |
| | | ZNF727 | P | THBD | SLC4A1 |
| | | RELN | ABCG1 | GZF1 | LRRC37A2 |
| | | PODXL | MIOX | MYL9 | ITGA3 |
| | | FGFR1 | IGFBP7 | TGM2 | AFMID |
| | | SULF1 | ENPEP | PCK1 | USP36 |
| | | GPT | SLC1A3 | ABCG1 | EMILIN2 |
| | | NR4A3 | ENC1 | MIOX | GREB1L |
| | | | F2R | FAM157A | DSG3 |
| | | | BHMT | IGFBP7 | ZNF69 |
| | | | DPYSL3 | BTC | NPHS1 |
| | | | SYNPO | ENPEP | WDR87 |
| | | | SPARC | SLC1A3 | APOC1 |
| | | | FOXQ1 | ENC1 | ZNF331 |
| | | | HLA-DRB5 | F2R | NAT8 |
| | | | CLIC5 | DPYSL3 | MRPL19 |
| | | | RP9 | SYNPO | ASTL |
| | | | ZNF727 | SPARC | SULT1C2 |
| | | | RELN | HLA-DRB5 | NR4A2 |
| | | | PODXL | CLIC5 | PLA2R1 |
| | | | SULF1 | RP9 | SCN2A |
| | | | GPT | ZNF727 | CERKL |
| | | | NTRK2 | RELN | COL3A1 |
| | | | NR4A3 | PODXL | HSPE1 |
| | | | GPSM1 | FGFR1 | SERPINE2 |
| | | | | SULF1 | UGT1A9 |
| | | | | TNFRSF11B | AGXT |
| | | | | GPT | THBD |
| | | | | NR4A3 | MYL9 |
| | | | | TNC | TGM2 |
| | | | | GPSM1 | PCK1 |
| | | | | FHL1 | ANKRD20A11P |
| | | | | | ABCG1 |
| | | | | | UPK3A |
| | | | | | MIOX |
| | | | | | CTDSPL |
| | | | | | DNAH12 |
| | | | | | HGD |
| | | | | | CASR |
| | | | | | SDHAP1 |
| | | | | | FAM157A |
| | | | | | IGFBP7 |
| | | | | | BTC |
| | | | | | ENPEP |
| | | | | | TNIP3 |
| | | | | | SLC1A3 |
| | | | | | MAP1B |
| | | | | | ENC1 |
| | | | | | F2R |
| | | | | | BHMT |
| | | | | | DPYSL3 |
| | | | | | SPINK1 |
| | | | | | SYNPO |
| | | | | | SPARC |
| | | | | | FOXQ1 |
| | | | | | HIST1H2AJ |
| | | | | | CLIC5 |
| | | | | | TPBG |
| | | | | | RGS17 |
| | | | | | RP9 |
| | | | | | ZNF727 |
| | | | | | RELN |
| | | | | | PODXL |
| | | | | | FGFR1 |
| | | | | | SULF1 |
| | | | | | TNFRSF11B |
| | | | | | GPT |
| | | | | | CD274 |
| | | | | | MOB3B |
| | | | | | NTRK2 |
| | | | | | NR4A3 |
| | | | | | TNC |
| | | | | | GPSM1 |
| | | | | | MIR6087 |
| | | | | | FHL1 |

TABLE 4
Differential gene expression markers for assessment of DN vs. CKD (female subjects)

| Number of Genes | 38 | 53 | 59 | 76 | 110 |
|---|---|---|---|---|---|
| Percentage of | 95.98% | 99.07 | 99.40% | 99.70% | 100% |
| Positive Samples | | | | | |
| with a Score | | | | | |
| of >0.90 and | | | | | |
| Negative Samples | | | | | |
| with a Score | | | | | |
| of <0.1 | | | | | |
| Gene List | ISG15 | ISG15 | ISG15 | ISG15 | ISG15 |
| | VCAM1 | IFI44L | IFI44L | PLA2G2F | PLA2G2F |
| | CIART | VCAM1 | VCAM1 | ERICH3 | ERICH3 |
| | NES | CIART | CIART | IFI44L | IFI44L |
| | NPHS2 | NES | NES | VCAM1 | IFI44 |
| | IFIT1 | NPHS2 | NPHS2 | CIART | F3 |
| | WT1 | IGFN1 | IGFN1 | LCE3D | VCAM1 |
| | IL18BP | IFIT1 | IFIT1 | NES | CIART |
| | C3AR1 | WT1 | WT1 | NPHS2 | LCE3D |
| | A2M | IL18BP | IL18BP | PKP1 | NES |
| | PTPRQ | C3AR1 | C3AR1 | PIGR | NPHS2 |
| | DHRS2 | PTPRO | A2M | IFIT1 | IGFN1 |
| | JAG2 | PTPRQ | PTPRO | WT1 | PKP1 |
| | GOLGA6L2 | DHRS2 | PTPRQ | IL18BP | PIGR |
| | SLC12A3 | JAG2 | DHRS2 | C3AR1 | HIST3H2BB |
| | SLC4A1 | GOLGA6L2 | C14orf37 | A2M | ITIH5 |
| | ARHGAP28 | SLC12A3 | TMEM30B | PTPRO | IFIT1 |
| | ANKRD20A | LOC388282 | JAG2 | ESPL1 | HTRA1 |
| | 5P | SLC4A1 | SLC12A3 | PTPRQ | WT1 |
| | PLIN4 | ARHGAP28 | LOC388282 | FREM2 | C11orf96 |
| | NPHS1 | ANKRD20A5 | NOS2 | ERICH6B | MS4A4A |
| | KLK5 | P | SLC4A1 | DHRS2 | IL18BP |
| | KLK7 | PLIN4 | ARHGAP28 | C14orf37 | KCNJ5 |
| | ID2 | ALKBH7 | ANKRD20A5P | TMEM30B | SLC2A14 |
| | PLA2R1 | NPHS1 | PLIN4 | JAG2 | C3AR1 |
| | FN1 | KLK5 | UPK1A | GOLGA6L2 | A2M |
| | SERPINE2 | KLK7 | NPHS1 | TMEM265 | PTPRO |
| | ANKRD20A | ID2 | KLK5 | SLC12A3 | KRT3 |
| | 11P | IL36RN | KLK7 | LOC388282 | ESPL1 |
| | ABCG1 | PLA2R1 | ID2 | NOS2 | PTPRQ |
| | CECR2 | FN1 | EHD3 | SLC4A1 | ERICH6B |
| | UPK3A | SERPINE2 | CXCR4 | ARHGAP28 | CDH24 |
| | FOXQ1 | ANKRD20A1 | PLA2R1 | ANKRD20A5P | DHRS2 |
| | GABBR1 | 1P | FN1 | PLIN4 | C14orf37 |
| | TREM2 | ABCG1 | SERPINE2 | ALKBH7 | TMEM30B |
| | CLIC5 | CECR2 | ANKRD20A11 | NPHS1 | JAG2 |
| | TPBG | ZNF74 | P | KLK5 | GOLGA6L2 |
| | PODXL | UPK3A | ABCG1 | KLK7 | SPTBN5 |
| | C8orf4 | TNNC1 | CECR2 | KLK8 | C15orf59 |
| | SULF1 | PLA1A | ZNF74 | ZNF71 | GPT2 |
| | | NWD2 | UPK3A | ID2 | SLC12A3 |
| | | HERC5 | TNNC1 | IL36RN | LOC388282 |
| | | MAP1B | NWD2 | CXCR4 | NOS2 |
| | | SPOCK1 | HERC5 | PLA2R1 | KRT24 |
| | | FOXQ1 | MAP1B | FN1 | SLC4A1 |
| | | GABBR1 | LHFPL2 | SERPINE2 | ARHGAP28 |
| | | TREM2 | SPOCK1 | ANKRD20A11 | ANKRD20A5P |
| | | CLIC5 | FOXQ1 | P | SLC14A2 |
| | | TPBG | GABBR1 | ABCG1 | PLIN4 |
| | | PODXL | TREM2 | CECR2 | ALKBH7 |
| | | LPL | CLIC5 | ZNF74 | FXYD3 |
| | | C8orf4 | TPBG | UPK3A | CD22 |
| | | SULF1 | RELN | TNNC1 | UPK1A |
| | | ZNF658 | PODXL | PLA1A | NPHS1 |
| | | ORM1 | DLC1 | FAM157A | KLK5 |
| | | | LPL | NWD2 | KLK7 |
| | | | C8orf4 | RASGEF1B | KLK8 |
| | | | SULF1 | HERC5 | ZNF71 |
| | | | ZNF658 | ENPEP | ZNF530 |
| | | | GPSM1 | TMEM144 | ID2 |
| | | | | F2R | EHD3 |
| | | | | SPARC | IL36RN |
| | | | | HIST1H4I | CXCR4 |
| | | | | GABBR1 | NR4A2 |
| | | | | TREM2 | PLA2R1 |
| | | | | CLIC5 | TFPI |
| | | | | TPBG | FN1 |
| | | | | HGF | SERPINE2 |
| | | | | PODXL | SIGLEC1 |
| | | | | DLC1 | ANKRD20A11P |
| | | | | LPL | ABCG1 |
| | | | | C8orf4 | CECR2 |
| | | | | SULF1 | ZNF74 |
| | | | | LAPTM4B | UPK3A |
| | | | | GPT | TNNC1 |
| | | | | ZNF658 | PLA1A |
| | | | | ORM1 | ALG1L |
| | | | | | FAM157A |
| | | | | | RASGEF1B |
| | | | | | HERC5 |
| | | | | | ADH1C |
| | | | | | ENPEP |
| | | | | | PRDM5 |
| | | | | | CTSO |
| | | | | | TMEM144 |
| | | | | | MAP1B |
| | | | | | LHFPL2 |
| | | | | | SPOCK1 |
| | | | | | SYNPO |
| | | | | | SPARC |
| | | | | | FOXQ1 |
| | | | | | HIST1H4I |
| | | | | | GABBR1 |
| | | | | | TREM2 |
| | | | | | CLIC5 |
| | | | | | TPBG |
| | | | | | GPNMB |
| | | | | | HGF |
| | | | | | RELN |
| | | | | | PODXL |
| | | | | | GIMAP2 |
| | | | | | DLC1 |
| | | | | | LPL |
| | | | | | C8orf4 |
| | | | | | SULF1 |
| | | | | | LY6E |
| | | | | | TPM2 |
| | | | | | ZNF658 |
| | | | | | GPSM1 |
| | | | | | XPNPEP2 |

TABLE 5
Differential gene expression markers for
assessment of DN vs. NEG (male subjects)

| Number of Genes | 74 | 88 | 94 | 106 |
|---|---|---|---|---|
| Percentage of | 98.92% | 99.19% | 99.46% | 100% |
| Positive Samples | | | | |
| with a Score | | | | |
| of >0.90 and | | | | |
| Negative Samples | | | | |
| with a Score | | | | |
| of <0.1 | | | | |
| Gene List | SNORA44 | SNORA44 | SNORA44 | SNORA44 |
| | SLC6A9 | SLC6A9 | SLC6A9 | SLC6A9 |
| | VCAM1 | VCAM1 | VCAM1 | VCAM1 |
| | LCE3D | KCND3 | KCND3 | KCND3 |
| | SPRR2D | LCE3D | LCE3D | LCE3D |
| | SPRR2E | SPRR2D | SPRR2D | SPRR2D |
| | SPRR2F | SPRR2A | SPRR2E | SPRR2A |
| | SNORA80E | SPRR2E | SPRR2F | SPRR2E |
| | NES | SPRR2F | SNORA80E | SPRR2F |
| | PAPPA2 | SNORA80E | NES | SNORA80E |
| | NPHS2 | NES | PAPPA2 | NES |
| | PRG4 | PAPPA2 | NPHS2 | PAPPA2 |
| | KIF14 | NPHS2 | PRG4 | NPHS2 |
| | ITIH5 | PRG4 | KIF14 | PRG4 |
| | SPOCK2 | KIF14 | ITIH5 | KIF14 |
| | HTRA1 | ITIH5 | SPOCK2 | ITIH5 |
| | WT1 | SPOCK2 | RBP4 | SPOCK2 |
| | TAGLN | HTRA1 | HTRA1 | RBP4 |
| | PTPRO | WT1 | WT1 | PNLIPRP3 |
| | KRT6A | GLYATL1 | TAGLN | HTRA1 |
| | PTPRQ | TAGLN | PTPRO | WT1 |
| | SNORA49 | PTPRO | KRT6A | GLYATL1 |
| | COL4A1 | KRT6A | PTPRQ | TAGLN |
| | COL4A2 | PTPRQ | SNORA49 | PTPRO |
| | NID2 | SNORA49 | COL4A1 | KRT6A |
| | C14orf37 | COL4A1 | COL4A2 | PTPRQ |
| | CORO2B | COL4A2 | NID2 | SNORA49 |
| | CYP1A1 | NID2 | C14orf37 | COL4A1 |
| | ARRDC4 | C14orf37 | ATP10A | COL4A2 |
| | UMOD | ATP10A | CORO2B | NID2 |
| | SLC12A3 | CORO2B | CYP1A1 | C14orf37 |
| | SNORA48 | CYP1A1 | ARRDC4 | ATP10A |
| | SCARNA21 | ARRDC4 | UMOD | CORO2B |
| | CDH2 | NOMO3 | SLC12A3 | CYP1A1 |
| | LIPG | UMOD | SNORA48 | ARRDC4 |
| | SERPINB4 | SLC12A3 | SCARNA21 | NOMO3 |
| | NPHS1 | SNORA48 | ARHGAP28 | UMOD |
| | KLK4 | SCARNA21 | CDH2 | SLC12A3 |
| | NAT8 | KRT16 | LIPG | SNORA48 |
| | PLA2R1 | CDH2 | SERPINB4 | SCARNA21 |
| | LRP2 | LIPG | SERPINB3 | CCL5 |
| | COL3A1 | SERPINB4 | NPHS1 | KRT14 |
| | UGT1A9 | NPHS1 | KLK4 | KRT16 |
| | HJURP | KLK4 | PXDN | ARHGAP28 |
| | BMP2 | PXDN | GREB1 | CDH2 |
| | MYL9 | NAT8 | NAT8 | LIPG |
| | NEFH | IL36A | GNLY | SNORA37 |
| | CLSTN2 | PLA2R1 | IL36A | SERPINB4 |
| | PXYLP1 | SCN9A | PLA2R1 | SERPINB3 |
| | SNORA63 | LRP2 | SCN9A | NPHS1 |
| | SPON2 | COL3A1 | LRP2 | KLK4 |
| | IGFBP7 | UGT1A9 | COL3A1 | PXDN |
| | ENPEP | HJURP | SLC23A3 | GREB1 |
| | PDLIM3 | BMP2 | SCARNA6 | NAT8 |
| | CDH6 | MYL9 | UGT1A9 | CXCR4 |
| | F2R | PABPC1L | HJURP | PLA2R1 |
| | SPOCK1 | NEFH | BMP2 | SCN9A |
| | SLC23A1 | CLSTN2 | MYL9 | LRP2 |
| | DPYSL3 | PXYLP1 | PABPC1L | COL3A1 |
| | SPARC | SNORA63 | NEFH | FN1 |
| | SNORA38 | SPON2 | LIF | SLC23A3 |
| | CLIC5 | IGFBP7 | CLSTN2 | SCARNA6 |
| | ENPP1 | ENPEP | PXYLP1 | UGT1A9 |
| | AEBP1 | CDH6 | SNORA63 | HJURP |
| | COL1A2 | MAP1B | SPON2 | BMP2 |
| | SGCE | F2R | IGFBP7 | MYL9 |
| | RELN | SPOCK1 | ENPEP | PABPC1L |
| | PODXL | SLC23A1 | PDLIM3 | NEFH |
| | ADAMDEC | DPYSL3 | CDH6 | LIF |
| | 1 | SPARC | F2R | TCN2 |
| | SULF1 | HAVCR1 | SPOCK1 | ALG1L |
| | GPT | SLC17A3 | SLC23A1 | CLSTN2 |
| | PTPRD | SNORA38 | DPYSL3 | PXYLP1 |
| | TMOD1 | CLIC5 | SPARC | SNORA63 |
| | FHL1 | ENPP1 | HAVCR1 | SPON2 |
| | | FKBP9 | SLC17A3 | IGFBP7 |
| | | AEBP1 | SNORA38 | ALB |
| | | COL1A2 | CLIC5 | ENPEP |
| | | SGCE | ENPP1 | GUCY1B3 |
| | | RELN | FKBP9 | PDLIM3 |
| | | SLC26A4 | AEBP1 | CDH6 |
| | | PODXL | COL1A2 | F2R |
| | | ADAMDEC1 | SGCE | SPOCK1 |
| | | SULF1 | RELN | SLC23A1 |
| | | GPT | SLC26A4 | DPYSL3 |
| | | PTPRD | PODXL | SPARC |
| | | TMOD1 | ADAMDEC1 | HAVCR1 |
| | | FHL1 | SULF1 | SLC17A3 |
| | | | GPT | SNORA38 |
| | | | PTPRD | CLIC5 |
| | | | ALDH1A1 | ENPP1 |
| | | | TMOD1 | FKBP9 |
| | | | XIST | AEBP1 |
| | | | FHL1 | COL1A2 |
| | | | | SGCE |
| | | | | RELN |
| | | | | SLC26A4 |
| | | | | PODXL |
| | | | | ADAMDEC1 |
| | | | | SULF1 |
| | | | | GPT |
| | | | | PTPRD |
| | | | | TMOD1 |
| | | | | TSIX |
| | | | | XIST |
| | | | | FHL1 |

TABLE 6
Differential gene expression markers for assessment of DN vs. CKD (male subjects)

| Number of Genes | 50 | 60 | 70 | 106 | 134 |
|---|---|---|---|---|---|
| Percentage of | 94.08% | 94.65% | 99.15% | 99.43% | 100% |
| Positive Samples | | | | | |
| with a Score | | | | | |
| of >0.90 and | | | | | |
| Negative Samples | | | | | |
| with a Score | | | | | |
| of <0.1 | | | | | |
| Gene List | SNORA61 | SYTL1 | SYTL1 | SYTL1 | RUNX3 |
| | SNORA44 | SNORA61 | SNORA61 | SNORA61 | SYTL1 |
| | VCAM1 | SNORA44 | SNORA44 | SNORA44 | SCARNA1 |
| | HIST2H2BE | VCAM1 | GBP6 | IFI44 | SNORA61 |
| | NES | HIST2H2BE | VCAM1 | GBP6 | SNORA44 |
| | NPHS2 | NES | FCGR1B | CDC14A | IFI44 |
| | PRG4 | NPHS2 | HIST2H2BE | VCAM1 | GBP6 |
| | FCMR | PRG4 | NES | FCGR1B | CDC14A |
| | ITIH5 | FCMR | NPHS2 | HIST2H2BE | VCAM1 |
| | HTRA1 | ITIH5 | PRG4 | TCHH | FCGR1B |
| | WT1 | HTRA1 | FCMR | NES | HIST2H2BF |
| | CD3E | WT1 | ITIH5 | NPHS2 | HIST2H2BE |
| | KCNJ5 | SNORA8 | HTRA1 | PRG4 | CTSK |
| | PTPRO | CD3E | WT1 | FCMR | LCE3D |
| | PTPRQ | KCNJ5 | SNORA8 | HIST3H2BB | NES |
| | SNORA27 | PTPRO | CD3E | ITIH5 | NPHS2 |
| | C14orf37 | PTPRQ | KCNJ5 | HTRA1 | PRG4 |
| | FBN1 | SNORA27 | PTPRO | MUC15 | FCMR |
| | ARRDC4 | DACH1 | KRT6B | WT1 | HIST3H2BB |
| | OTOA | C14orf37 | PTPRQ | SNORA8 | ITIH5 |
| | SLC12A3 | FBN1 | SNORA27 | CD3E | SLC29A3 |
| | CCL5 | ARRDC4 | DACH1 | KCNJ5 | BAG3 |
| | ARHGAP28 | AMDHD2 | C14orf37 | PTPRO | HTRA1 |
| | NPHS1 | OTOA | KIF26A | KRT6B | WT1 |
| | KLK4 | SLC12A3 | FBN1 | PTPRQ | C11orf84 |
| | KLK6 | CCL5 | ARRDC4 | GJB6 | RELT |
| | KLK7 | ARHGAP28 | OTOA | SNORA27 | SNORD15B |
| | GREB1 | NPHS1 | MT1E | KIAA0226L | SNORA8 |
| | PLA2R1 | PSG4 | SLC12A3 | DACH1 | CD3E |
| | COL3A1 | KLK4 | CCL5 | FOXA1 | KCNJ5 |
| | ADAMTS1 | KLK6 | KRT16 | C14orf37 | TMEM52B |
| | NEFH | KLK7 | ARHGAP28 | KIF26A | PTPRO |
| | LGALS2 | GREB1 | NPHS1 | FBN1 | KRT6B |
| | B3GALNT1 | ZFP36L2 | PSG4 | ARRDC4 | KRT6A |
| | CPZ | CD8A | NAPSB | AMDHD2 | PTPRQ |
| | NPNT | GYPC | KLK4 | OTOA | MYBPC1 |
| | ENPEP | PLA2R1 | KLK5 | MT1E | HPD |
| | SPOCK1 | COL3A1 | KLK6 | MT1G | GJB6 |
| | SPARC | CEBPB | KLK7 | SLC12A3 | SNORA27 |
| | GMPR | ADAMTS1 | GREB1 | RPH3AL | KIAA0226L |
| | HIST1H1D | NEFH | CD8A | CCL5 | DACH1 |
| | CLIC5 | B3GALNT1 | PLA2R1 | KRT16 | FOXA1 |
| | AGR2 | CPZ | COL3A1 | CD300C | C14orf37 |
| | COL1A2 | NPNT | D2HGDH | ARHGAP28 | FBN1 |
| | RELN | ENPEP | CEBPB | CDH2 | ARRDC4 |
| | SLC26A4 | SPOCK1 | ADAMTS1 | SERPINB2 | AMDHD2 |
| | PODXL | SPARC | NEFH | PRAM1 | OTOA |
| | ADAMDEC | GMPR | LGALS2 | ACP5 | MT1E |
| | 1 | HIST1H1D | B3GALNT1 | NPHS1 | MT1G |
| | SULF1 | CLIC5 | CPZ | PSG4 | SLC12A3 |
| | FHL1 | LAMA4 | NPNT | FKRP | RPH3AL |
| | | HGF | ENPEP | NAPSB | ZNF18 |
| | | COL1A2 | SPOCK1 | KLK4 | CCL5 |
| | | RELN | SPARC | KLK5 | KRT16 |
| | | SLC26A4 | GMPR | KLK6 | ARHGAP28 |
| | | PODXL | HIST1H1D | KLK7 | CDH2 |
| | | RARRES2 | HIST1H2BM | KLK8 | SERPINB2 |
| | | ADAMDEC1 | HLA-L | GREB1 | PRAM1 |
| | | SULF1 | CLIC5 | ZFP36L2 | ACP5 |
| | | FHL1 | AGR2 | CD8A | IER2 |
| | | | SNORA22 | GYPC | ZNF714 |
| | | | COL1A2 | PLA2R1 | CCNE1 |
| | | | RELN | COL3A1 | CEBPA |
| | | | SLC26A4 | D2HGDH | NPHS1 |
| | | | PODXL | SIRPB1 | PSG4 |
| | | | RARRES2 | CEBPB | FKRP |
| | | | ADAMDEC1 | ADAMTS1 | NAPSB |
| | | | ADAM32 | NEFH | KLK4 |
| | | | SULF1 | LGALS2 | KLK5 |
| | | | FHL1 | TNNC1 | KLK6 |
| | | | | ABI3BP | KLK7 |
| | | | | GATA2 | KLK8 |
| | | | | ACPP | ZNF677 |
| | | | | B3GALNT1 | GREB1 |
| | | | | CPZ | ADCY3 |
| | | | | SNORA26 | ZFP36L2 |
| | | | | NPNT | CD8A |
| | | | | ENPEP | GYPC |
| | | | | SPOCK1 | PLA2R1 |
| | | | | SPARC | SCN2A |
| | | | | SERPINB9 | COL3A1 |
| | | | | GMPR | VIL1 |
| | | | | HIST1H1D | D2HGDH |
| | | | | HIST1H2BM | MAFB |
| | | | | HLA-L | PABPC1L |
| | | | | CLIC5 | CEBPB |
| | | | | PLA2G7 | ADAMTS1 |
| | | | | LAMA4 | CRYAA |
| | | | | TAGAP | NEFH |
| | | | | AGR2 | LGALS2 |
| | | | | SNORA22 | GPX1 |
| | | | | HGF | ABI3BP |
| | | | | TFPI2 | GATA2 |
| | | | | COL1A2 | ACPP |
| | | | | PEG10 | ESYT3 |
| | | | | RELN | B3GALNT1 |
| | | | | SLC26A4 | CPZ |
| | | | | PODXL | SNORA26 |
| | | | | RARRES2 | NPNT |
| | | | | DLC1 | ENPEP |
| | | | | ADAMDEC1 | SPOCK1 |
| | | | | HTRA4 | SNORA74A |
| | | | | ADAM32 | SPARC |
| | | | | CEBPD | SERPINB9 |
| | | | | SULF1 | GMPR |
| | | | | FHL1 | HIST1H1D |
| | | | | | HIST1H2BM |
| | | | | | HLA-L |
| | | | | | CLIC5 |
| | | | | | PLA2G7 |
| | | | | | LAMA4 |
| | | | | | TAGAP |
| | | | | | AGR2 |
| | | | | | GPNMB |
| | | | | | CREB5 |
| | | | | | TARP |
| | | | | | SNORA22 |
| | | | | | TFPI2 |
| | | | | | COL1A2 |
| | | | | | RELN |
| | | | | | SLC26A4 |
| | | | | | PODXL |
| | | | | | RARRES2 |
| | | | | | DLC1 |
| | | | | | ADAMDEC1 |
| | | | | | FGFR1 |
| | | | | | HTRA4 |
| | | | | | ADAM32 |
| | | | | | SULF1 |
| | | | | | TNFRSF11B |
| | | | | | FOXE1 |
| | | | | | ZNF711 |
| | | | | | WBP5 |
| | | | | | FHL1 |

As described above, using methods and systems of the present disclosure, urine samples were analyzed from subjects to non-invasively assess diabetic nephropathy (DN). Notably, several improvements were leveraged to obtain excellent performance results, including: unique DN selection criteria, gender-specific DN gene signatures, a two-biomarker DN assessment approach, the use of 4 gene signatures (combining the gender-specific signatures and two-biomarker approaches), and use of the Recursive Feature Elimination classifier and the number of features used. For example, 2 gene signatures may be used for a male subject (a male-specific DN vs. NEG gene signature, and a male-specific DN vs. CKD gene signature), while 2 different gene signatures may be used for a female subject (a female-specific DN vs. NEG gene signature, and a female-specific DN vs. CKD gene signature).

Alternatively to using the two-biomarker scoring approach (e.g., using a first DN vs. NEG score and a second DN vs. CKD score), a multi-class classifier may be directly trained using a training dataset including positive cases and negative controls for two or more kidney diseases or disorders, such as DN and another kidney disease or disorder (e.g., DN and CKD), by using the gene markers listed in Table 3, Table 4, Table 5, and Table 6 as input features for the multi-class classifier. The multi-class classifier may be trained to distinguish between three or more cases: positive for a first kidney disease or disorder, positive for a second kidney disease or disorder, and negative (e.g., having no kidney disease or disorder).

Patients with diabetic nephropathy may benefit from treatments being developed using regenerative medicine. These techniques may help reverse or slow kidney damage caused by the disease. For example, if a patient's diabetes can be cured by a treatment such as pancreas islet cell transplant or stem cell therapy, their kidney function may improve. In addition, novel therapies such as stem cell therapies and new medications may be developed for use in treating diabetic nephropathy.

Other types of kidney damage (e.g., non-diabetic nephropathy) may be identified using a two-biomarker approach by modifying negative samples (e.g., in the NO group). For example, if the kidney disease-vs.-negative score for a given kidney disease increases significantly (e.g., by more than 0.1) as a result of replacing nicotine-dependent subjects in the NO group with non-smoker subjects, then it may be likely that albuminuria may be a result of nicotine use. This approach may be applied to identify a variety of kidney diseases and disorders, such as hypertensive nephropathy, IgA nephropathy, membranous nephropathy, minimal change disease, focal segmental glomerulosclerosis (FSGS), NSAIDs induced nephrotoxicity, thin basement membrane nephropathy, amyloidosis, ANCA vasculitis related to endocarditis and other infections, cardiorenal syndrome, IgG4 nephropathy, interstitial nephritis, lithium nephrotoxicity, lupus nephritis, multiple myeloma, polycystic kidney disease, pyelonephritis (kidney infection), renal artery stenosis, renal cyst, rheumatoid arthritis-associated renal disease, and kidney stones.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for processing or analyzing a bodily sample of a subject, comprising:
(a) analyzing said bodily sample to yield a data set comprising a set of levels of gene expression products in said bodily sample, which set of levels of gene expression products correspond to a set of genes associated with a kidney disease or disorder;
(b) computer processing said data set from (a) to determine a presence, an absence, an elevated risk, or a decreased risk of said kidney disease or disorder in said subject at an accuracy of at least about 80%, as determined by a percentage of independent test subjects that are correctly identified or classified as either having or not having the kidney disease or disorder; and
(c) electronically outputting a report that identifies said presence, said absence, said elevated risk, or said decreased risk of said kidney disease or disorder in said subject determined in (b).

2. The method of claim 1, wherein said bodily sample is selected from the group consisting of: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample.

3. The method of claim 2, wherein said bodily sample is said urine sample.

4. (canceled)

5. The method of claim 1, wherein (a) comprises reverse transcribing ribonucleic acid (RNA) molecules obtained or derived from said bodily sample to yield complementary deoxyribonucleic acid (cDNA) molecules, and sequencing at least a portion of said cDNA molecules to yield said data set, wherein said data set comprises sequencing reads.

6. (canceled)

7. (canceled)

8. (canceled)

9. The method of claim 5, wherein (a) comprises selectively enriching and amplifying at least a portion of said cDNA molecules for a set of genomic loci associated with said kidney disease or disorder.

10. (canceled)

11. The method of claim 5, wherein (a) comprises aligning at least a portion of said sequencing reads to a human reference genome, generating counts of gene transcripts from said aligned sequencing reads, and normalizing said counts to generate normalized counts of gene transcripts.

12. (canceled)

13. (canceled)

14. (canceled)

15. The method of claim 1, wherein said kidney disease or disorder is selected from the group consisting of: early-stage kidney disease, mid-stage kidney disease, late-stage kidney disease, end-stage kidney disease, asymptomatic kidney disease, diabetic nephropathy, hypertensive nephropathy, IgA nephropathy, membranous nephropathy, minimal change disease, focal segmental glomerulosclerosis (FSGS), NSAIDs induced nephrotoxicity, thin basement membrane nephropathy, amyloidosis, ANCA vasculitis related to endocarditis and other infections, cardiorenal syndrome, IgG4 nephropathy, interstitial nephritis, lithium nephrotoxicity, lupus nephritis, multiple myeloma, polycystic kidney disease, pyelonephritis, renal artery stenosis, renal cyst, rheumatoid arthritis-associated renal disease, and kidney stone.

16. The method of claim 15, wherein said kidney disease or disorder is diabetic nephropathy.

17. The method of claim 16, wherein said diabetic nephropathy is early-stage diabetic nephropathy.

18. (canceled)

19. The method of claim 16, wherein said set of genes comprises at least one gene selected from the group consisting of genes listed in Table 3, genes listed in Table 4, genes listed in Table 5, and genes listed in Table 6.

20. The method of claim 1, wherein (b) comprises using a trained machine learning algorithm to process said data set.

21. (canceled)

22. The method of claim 20, wherein said trained machine learning algorithm is selected from the group consisting of: a support vector machine (SVM), a naïve Bayes classification, a linear regression, a quantile regression, a logistic regression, a nonlinear regression, a random forest, a neural network, an ensemble learning method, a boosting algorithm, an AdaBoost algorithm, a recursive feature elimination algorithm (RFE), and any combination thereof.

23. The method of claim 22, wherein said trained machine learning algorithm comprises said recursive feature elimination (RFE) algorithm.

24. The method of claim 20, wherein said trained machine learning algorithm is trained with a plurality of training samples comprising a first set of bodily samples from subjects having said kidney disease or disorder and a second set of bodily samples from subjects having no kidney disease or disorder, wherein said first set of bodily samples and said second set of bodily samples are different from said bodily sample of said subject.

25. The method of claim 20, wherein said trained machine learning algorithm is trained with a plurality of training samples comprising a first set of bodily samples from subjects having said kidney disease or disorder and a second set of bodily samples from subjects having other types of kidney disease or disorder, wherein said first set of bodily samples and said second set of bodily samples are different from said bodily sample of said subject.

26. The method of claim 1, wherein (b) comprises comparing said set of levels of gene expression products to a reference.

27. (canceled)

28. (canceled)

29. The method of claim 1, further comprising detecting said presence, said absence, said elevated risk, or said decreased risk of said kidney disease or disorder in said subject at a sensitivity and a specificity of at least about 80%.

30.-35. (canceled)

36. The method of claim 1, further comprising providing a clinical intervention for said subject based at least in part on said presence or said elevated risk of said kidney disease or disorder determined in (b).

37. The method of claim 36, wherein said clinical intervention is selected from the group consisting of: a drug treatment, intensive glycemic control, high blood pressure control, lower high cholesterol, foster bone health, diet control, lifestyle changes, weight loss, exercise, tobacco cessation, manage alcohol intake, reduce/quit drugs of abuse, and avoiding NSAIDs.

38. (canceled)

39. The method of claim 1, wherein (b) comprises analyzing a first set of genes that differentially distinguishes between a first kidney disease or disorder and negative (NEG) subjects who do not have an overt renal manifestation, and a second set of genes that differentially distinguishes between said first kidney disease or disorder and a second kidney disease or disorder.

40. The method of claim 39, wherein (b) comprises analyzing a first set of genes that differentially distinguishes between diabetic nephropathy (DN) and negative (NEG) subjects who do not have an overt renal manifestation, and a second set of genes that differentially distinguishes between DN and other chronic kidney diseases (CKD).

41. The method of claim 40, wherein said first set of genes is selected from the group of genes listed in Table 3 and Table 5, and wherein said second set of genes is selected from the group of genes listed in Table 4 and Table 6.

42. The method of claim 40, wherein (b) comprises generating a first DN vs. NEG score based on said first set of genes and a second DN vs. CKD score based on said second set of genes.

43. The method of claim 42, wherein said first DN vs. NEG score is indicative of glomerular injury when greater than 0.5, or is indicative of tubular injury when less than 0.5.

44. The method of claim 1, wherein (b) comprises analyzing different male-specific or female-specific sets of genes based on a gender of said subject.

45. The method of claim 1, further comprising analyzing bodily samples of said subject at two or more different time points to yield two or more data sets, and computer processing said two or more data sets to determine said presence, said absence, said elevated risk, or said decreased risk of said kidney disease or disorder or another type of kidney disease or disorder in said subject.

46.-184. (canceled)

185. The method of claim 1, further comprising removing at least a subset of negative (NEG) subjects who have a pre-determined characteristic, and optionally replacing with additional NEG subjects who do not have the pre-determined characteristic, to generate a modified set of NEG-X subjects, wherein X is the pre-determined characteristic.

186. The method of claim 185, wherein the pre-determined characteristic is subjects who are obese, are morbidly obese, are nicotine dependent, are alcohol dependent, are drugs-of-abuse dependent, have kidney stone, have severe hypertension, have urinary tract infection, have heart diseases, have hepatitis B, have hepatitis C, have HIV, have psoriasis, have rheumatoid arthritis, or use NSAIDs.

187. The method of claim 185, wherein, if a DN vs. NEG-X score is much higher than the DN vs. NEG score, that is indicative of the subject having kidney damage as a result of the pre-determined characteristic X.

188. The method of claim 1, further comprising removing at least a subset of other chronic kidney disease (CKD) subjects who have a pre-determined characteristic, and optionally replacing with additional CKD subjects who do not have the pre-determined characteristic, to generate a modified set of CKD-Y subjects, wherein Y is the pre-determined characteristic.

189. The method of claim 188, wherein the pre-determined characteristic is subjects who are obese, are morbidly obese, are nicotine dependent, are alcohol dependent, are drugs-of-abuse dependent, have kidney stone, have severe hypertension, have urinary tract infection, have heart diseases, have hepatitis B, have hepatitis C, have HIV, have psoriasis, have rheumatoid arthritis, use NSAIDs, have IgA nephropathy, have membranous nephropathy, have minimal change disease, have focal segmental glomerulosclerosis (FSGS), have thin basement membrane nephropathy, have amyloidosis, have ANCA vasculitis related to endocarditis and other infections, have cardiorenal syndrome, have IgG4 nephropathy, have interstitial nephritis, have lithium nephrotoxicity, have lupus nephritis, have multiple myeloma, have polycystic kidney disease, have pyelonephritis (kidney infection), have renal artery stenosis, have renal cyst, or have rheumatoid arthritis-associated renal disease.

190. The method of claim 188, wherein, if a DN vs. CKD-Y score is much higher than the DN vs. CKD score, that is indicative of the subject having kidney damage as a result of the pre-determined characteristic Y.
